(19) United States
(12) Patent Application Publication
(10) Pub. No.: US 2026/0112494 A1
(43) Pub. Date: Apr. 23, 2026

(54) SYSTEMS AND METHODS FOR GENERATING A DIABETIC STATUS MODEL USING VOICE

(71) Applicant: KVI BRAVE FUND I INC., Toronto (CA)

(72) Inventors: Yan FOSSAT, Toronto (CA); Jaycee Morgan KAUFMAN, Toronto (CA); Jouhyun Clare JEON, Toronto (CA); Anirudh THOMMANDRAM, Toronto (CA)

(73) Assignee: KVI BRAVE FUND I INC., Toronto (CA)

(21) Appl. No.: 19/423,110

(22) Filed: Dec. 17, 2025

Related U.S. Application Data
(63) Continuation of application No. PCT/CA2024/051179, filed on Sep. 10, 2024.
Application No. PCT/CA2024/051179 is a continuation of application No. 18/244,400, filed on Sep. 11, 2023.

(51) Int. Cl. G16H 50/20; G10L 15/02

(52) U.S. Cl. CPC G16H 50/20; G10L 15/02

(57) ABSTRACT

Provided are methods and systems for generating a type-II (T2DM) diabetic status prediction model, the method comprising: providing, at a memory: a plurality of diabetic status labels identifying a corresponding diabetic status for a plurality of training subjects; and a plurality of voice samples collected from the plurality of training subjects at a plurality of time points, each of the voice samples in the plurality of voice samples associated with a corresponding diabetic status label in the plurality of diabetic status labels; determining, at the processor, a plurality of voice feature values for a corresponding plurality of voice features for each of the voice samples in the plurality of voice samples; and generating at the processor, the diabetic status model based on the plurality of voice samples and the plurality of voice feature values.

### CROSS REFERENCE TO RELATED APPLICATIONS

This application claims priority to U.S. patent application Ser. No. 18/244,400 filed Sep. 11, 2023 and PCT patent application no. PCT/CA2024/051179 filed Sep. 10, 2024, the entire contents of which are hereby incorporated by reference.

### FIELD

The described embodiments relate to systems and methods for generating a diabetic status model and more specifically to systems, devices and methods for generating a type-2 diabetes mellitus (T2DM) status model using voice samples.

### BACKGROUND

Human voice is composed of complex signals that are tightly associated with physiological changes in body systems. Due to the depth of signals that can be analyzed, as well as the wide range of potential physiological dysfunction that manifest in voice signals, voice has quickly gained traction in healthcare and medical research. For example, it has been shown that thyroid hormone imbalance caused the hoarseness of voice, and affected larynx development (Hari Kumar et al., 2016). Unstable pitch and loudness were observed in patients with multiple sclerosis (Noffs et al., 2018). Other recent studies also demonstrated distinct voice characteristics that were associated with various pathological, neurological, and psychiatric disorders, such as congestive heart failure (Maor et al., 2020), Parkinson's disease (Vaicuknyas et al., 2017), Alzheimer's disease (Fraser et al., 2015), posttraumatic stress disorder (Marmar et al., 2019), and autism spectrum disorder (Bonneh et al., 2011). The human voice is now considered as an emerging biomarker, which is inherently non-invasive, low-cost, accessible, and easy monitor for health conditions in various real-life settings.

Diabetes has a high incidence (10.5% of population in 2018) and is one of the main causes of death in the United States (7th leading cause). In spite of such risks, screening undiagnosed patients is not conducted routinely, and thus about 50% of adult diabetes cases are estimated to be undiagnosed, globally (Beagley et al., 2014).

Type 2 diabetes mellitus (T2DM) is a chronic metabolic disorder characterized by impaired insulin action and elevated blood glucose levels. Its rising prevalence and significant impact on global health have gained substantial attention in recent years, prompting a push for proactive measures. An estimated 175 million individuals worldwide have undiagnosed diabetes, and the cumulative economic burden is estimated to reach nearly $2.1 trillion USD per year in 2030 (Bommer et al., 2018). Additionally, diabetes diagnosis is associated with an increased risk of mortality from cancer, renal disease, infections, liver disease, nervous system disorders, and chronic obstructive pulmonary disease (Harding et al., 2019). It is imperative to develop effective strategies for disease detection that can identify individuals earlier in the disease trajectory, allowing for timely interventions and alleviating the consequences on individuals and healthcare infrastructures.

Recently, voice has emerged as a promising candidate for pathology detection. Voice synthesis is a complex process that relies on the combined effects of the respiratory system, nervous system, and the larynx. Anything that affects these systems can influence the voice, whether it is perceptible audibly or detectable through computer analysis (Zhang, 2016). Traditionally, voice and speech were noted to be significantly different in diseases that were directly responsible for voice production, such as respiratory diseases and laryngeal pathologies. Specifically, asthma and pulmonary function can lead to changes to air flow, and reflux laryngitis, vocal cord paralysis, and vocal cord lesion can result in structural changes in the larynx, resulting in breathy, low-pitched, hoarse, strained, or fatigued voices (Alam et al., 2022, Rosen et al., 1998). Voice analysis evolved to identify other, less direct illnesses such as coronary artery disease and sleep apnea, which began to use acoustic features of the voice to identify changes (Sara et al., 2022, Roy et al., 2019). Today, voice analysis has successfully been used to detect psychological illnesses such as depression and cognitive function decline (López-de-Ipiña et al., 2020, Wang et al., 2019).

T2DM and sustained periods of high blood glucose have been associated with a variety of complications. Peripheral neuropathy, or the damage of nerves outside of the spinal cord, occurs as a result of high glucose levels, as can nephropathy and myopathy (i.e. the damage of nerve and muscle fibers, respectively) (Yagihashi et al., 2011, Ciarambino et al., 2022). Furthermore, T2DM has been linked to an increased prevalence of psychological disorders such as depression, anxiety, eating disorders and decreasing cognitive function (Ciarambino et al., 2022, Palomo-Osuna et al., 2022). As these complications have been linked to vocal changes, it stands to reason that T2DM itself may be able to be detected from the voice.

Previous studies in the field of voice analysis have focused primarily on identifying features that may be different between the T2DM and non-diabetic populations, with varying results. A study of 83 participants conducted in Thailand indicated that fundamental frequency decreases significantly in T2DM females when compared to non-diabetic females (Pinyopodjanard et al., 2021). However, this study did not identify any differences in males, a result confirmed by a previous study conducted in 2012 (Hamdan et al., 2012). On the other hand, a study conducted in 2021 on 51 diabetic patients indicated that individuals with T2DM had an increased absolute jitter value compared to their healthy controls, although the sample was not segmented into male and female categories for the analysis (Gölaç et al., 2022). Finally, voice analysis conducted on 177 voice samples in 2016 showed a decrease in all vocal parameters for T2DM females, and all vocal parameters for T2DM males except absolute jitter and relative average perturbation (Chitkara et al., 2016). All previous studies analyzed sustained phonation of the vowel “a”. Although there have been some promising results, there is limited data on vocal changes between non-diabetic and T2DM individuals in age- and BMI-matched populations, and analysis has yet to be performed on a fixed sentence despite the reported success in determining glucose-related voice changes from spoken sentences and free speech (Sidorova et al., 2022).

There is a need for more advanced systems and methods for determining the association of voice signals with diabetic in healthy individuals, pre-diabetic individuals, and T2DM individuals and as a potential biomarker for disease.

### SUMMARY

Voice signal analysis is an emerging non-invasive technique to examine health conditions. The analysis of human voice data (including voice signal analysis) presents a technical computer-based problem which involves digital signal processing of the voice data. Analysis, including the use of predictive models, requires significant processing capabilities in order to determine biomarker signals and extract relevant information. The sheer number of available biomarker signals poses a challenge since the biomarkers must be efficiently selected in order to reduce processing overhead. Another challenge for voice signal analysis systems performing prediction is that they preferably function in real-time with the voice data collection and on a variety of different processing platforms and operate efficiently to deliver predictions and results to a user in a timely fashion.

In line with this pursuit, embodiments herein assess the feasibility of voice recording using mobile applications for prediction and detection of T2DM (Kaufman). Embodiments herein and associated Example results showed that voice biomarkers along with age and BMI are able to improve T2DM prediction with accuracy Of 0.89 for women and 0.86 for men (Kaufman). In Example #1, the study participants were tasked with recording their voices at least 6 times daily over a 2-week period. It became apparent that such a regimen could lead to participant burden and inconsistency in recordings. Therefore, further embodiments herein streamline this process by determining the 1) improved number of voice samples, and 2) improved recording schedule (frequency & timing), which is required to maintain T2DM diagnostic efficacy while reducing patient burden.

As set out in Example #1, a study was conducted to investigate the potential of voice analysis as a pre-screening or monitoring tool for Type 2 Diabetes Mellitus (T2DM) by examining the differences in voice recordings between non-diabetic individuals and those with T2DM. 267 participants diagnosed as non-diabetic or T2DM based on American Diabetes Association guidelines were recruited in India. Using a smartphone application, participants recorded a fixed phrase up to six times daily for two weeks, resulting in 18,465 recordings. Fourteen acoustic features were extracted from each recording to analyze differences between non-diabetic and T2DM individuals. Significant differences were found between voice recordings of non-diabetic and T2DM males and females, both in the entire dataset and in an age- and BMI-matched sample. The highest predictive accuracy was achieved by pitch (P<0.0001), pitch standard deviation (P<0.0001), and rap jitter (P=0.02) for females, and intensity (P<0.0001) and abq11 shimmer (P<0.0001) for males. Incorporating these features along with age and body mass index, the improved prediction models achieved accuracies of 0.78±0.28 for females and 0.73±0.08 for males through 10-fold cross validation in the age- and BMI-matched sample. Example #1 demonstrates that vocal changes occur in individuals with T2DM and can be distinguished from those without diabetes. Overall, voice analysis shows potential as a pre-screening or monitoring tool for T2DM, particularly when combined with other risk factors associated with the condition.

As set out in Example #2, an investigation was undertaken to determine a desirable number of voice samples, and recording schedule (frequency & timing), to maintain T2DM diagnostic efficacy while reducing patient burden. Using data from a cohort comprising 22 women and 56 men, the accuracy of previously developed models was evaluated and compared across various configurations of voice recordings and recording settings. Segmented linear regression was used to investigate the changes in model accuracy and Kendall's Tau measure of correlation was used to investigate the strength and direction of the relationship between recording settings and model accuracy. P-value of 0.05 were considered statistically significant. The results of Example #2 showed that using between 2 and 6 voice recordings yields improved diagnostic accuracy for T2DM. Moreover, it was observed that the day of voice recordings does not have a significant effect on model accuracy. However, adhering to recording within a single day demonstrated higher accuracy. Example #2 underscores improved voice recording settings to reduce patients burden while maintaining diagnostic efficacy.

In one aspect, there is provided a computer-implemented method for generating a type-II (T2DM) diabetic status prediction for a subject, the method comprising: providing, at a memory, a diabetic status prediction model; receiving, at a processor in communication with the memory, a voice sample from the subject; extracting, at the processor, at least one voice biomarker feature value from the voice sample for at least one predetermined voice biomarker feature; determining, at the processor, the type-II (T2DM) diabetic status prediction for the subject based on the at least one voice biomarker feature value and the diabetic status prediction model; and outputting, at an output device, the type-II (T2DM) diabetic status prediction for the subject or an output based on the diabetic status prediction.

In one or more embodiments, each of the at least one voice biomarker feature value may be selected from the group comprising: a statistical feature category, a shimmer feature category, and a jitter feature category.

In one or more embodiments, the statistical feature category may comprise a mean pitch feature value, a pitch standard deviation feature value, a mean intensity feature value, an intensity standard deviation feature value and a harmonic-to-noise ratio feature value; the shimmer feature category may comprise a localShimmer feature value, a localdbShimmer feature value, an apq3Shimmer feature value, an apq5Shimmer feature value, and an apq11Shimmer feature value; and the jitter feature category may comprise a localJitter feature value, a localabsJitter feature value, a rapJitter feature value and a ppq5Jitter feature value.

In one or more embodiments, the method may further comprise: preprocessing, at the processor, the voice sample by: storing, at a database in communication with the processor, a plurality of historical voice samples of the subject; and averaging the voice sample based on at least one of the plurality of historical voice samples of the subject.

In one or more embodiments, the voice sample may comprise a predetermined phrase vocalized by the subject; and the voice sample is received from a user device in network communication with the processor.

In one or more embodiments, the predetermined phrase may be displayed to the subject on a display device of the user device.

In one or more embodiments, the method may further comprise: transmitting, to the user device in network communication with the processor, the type-II (T2DM) diabetic status prediction for the subject, wherein the outputting of the diabetic status prediction for the subject occurs at the user device.

In one or more embodiments, the diabetic status prediction may comprise a categorical prediction.

In one or more embodiments, the categorical prediction may be one selected from the group of: a type-II (T2DM) diabetic category, and a normal category.

In one or more embodiments, the determining the diabetic status prediction for the subject may be based on at least one selected from the group of: vocal parameter data of the subject, age data of the subject, and Body Mass Index (BMI) data of the subject.

In one or more embodiments, the diabetic status prediction model may comprise at least one selected from the group of a Logistic Regression (LR) model, a Naïve Bayes (NB) model, and a Support Vector Machine (SVM) model.

In one or more embodiments, the diabetic status prediction model may comprise an ensemble model. This may include, averaging all the prediction probabilities within an individual, averaging the voice prediction results with the T2DM prevalence at the participant's age, averaging the voice prediction results with the T2DM prevalence at the participant's BMI, and/or a combination of these methods.

In a second aspect, there is provided a computer-implemented system for predicting a type-II (T2DM) diabetic status for a subject, the system comprising a processor and a memory in communication with the processor, the processor configured to provide the methods herein.

In a third aspect, there is provided a device for predicting a type-II (T2DM) diabetic status for a subject, the device comprising a processor and a memory in communication with the processor, the processor configured to provide the methods herein.

In a fourth aspect, there is provided a computer-implemented method for generating a diabetic status model for predicting a type-II (T2DM) diabetic status for a subject, the method comprising: providing, at a memory: a plurality of diabetic status labels, the plurality of diabetic status labels identifying a corresponding diabetic status for a plurality of training subjects; and a plurality of voice samples collected from the plurality of training subjects at a plurality of time points, each of the voice samples in the plurality of voice samples associated with a corresponding diabetic status label in the plurality of diabetic status labels; determining, at the processor, a plurality of voice feature values for a corresponding plurality of voice features for each of the voice samples in the plurality of voice samples; and generating at the processor, the diabetic status model based on the plurality of voice samples and the plurality of voice feature values.

In one or more embodiments, each of the at least one voice biomarker feature value may be selected from the group comprising: a statistical feature category, a shimmer feature category, and a jitter feature category.

In one or more embodiments, the statistical feature category may comprise a mean pitch feature value, a pitch standard deviation feature value, a mean intensity feature value, an intensity standard deviation feature value and a harmonic-to-noise ratio feature value; the shimmer feature category may comprise a localShimmer feature value, a localdbShimmer feature value, an apq3Shimmer feature value, an apq5Shimmer feature value, and an apq11Shimmer feature value; and the jitter feature category may comprise a localJitter feature value, a localabsJitter feature value, a rapJitter feature value and a ppq5Jitter feature value.

In one or more embodiments, the method may comprise: selecting, at the processor, a subset of voice feature values from the plurality of voice feature values for each voice sample based on a Cohen's d effect size of each voice feature value; and wherein the generating at the processor, the diabetic status model is based on the plurality of voice samples and the subset of voice feature values.

In one or more embodiments, the method may comprise: preprocessing, at the processor, each voice sample by averaging the voice sample based on at least one of the plurality of historical voice samples of the subject.

In one or more embodiments, the plurality of voice samples may comprise an age and BMI matched dataset comprising voice samples from an equal number of T2DM subjects and non-diabetic subjects.

In one or more embodiments, each voice sample may comprise a predetermined phrase vocalized by the corresponding subject.

In one or more embodiments, the predetermined phrase may be displayed to the subject on a display device of a user device and each voice sample may be recorded using an audio input device of the user device.

In one or more embodiments, the memory may further comprise: at least one selected from the group of: vocal parameter data for the plurality of subjects, age data of the plurality of subjects, and Body Mass Index (BMI) data of the plurality of subjects; and wherein the generating at the processor, the diabetic status model may be further based on the at least one selected from the group of: vocal parameter data for the plurality of subjects, age data of the plurality of subjects, and Body Mass Index (BMI) data of the plurality of subjects.

In one or more embodiments, the diabetic status prediction model may comprise at least one selected from the group of a Logistic Regression (LR) model, a Naïve Bayes (NB) model, and a Support Vector Machine (SVM) model.

In one or more embodiments, the diabetic status prediction model may comprise an ensemble model. This may include, averaging all the prediction probabilities within an individual, averaging the voice prediction results with the T2DM prevalence at the participant's age, averaging the voice prediction results with the T2DM prevalence at the participant's BMI, and/or a combination of these methods.

In a fifth aspect, there is provided a computer-implemented system for generating a diabetic status model for predicting a type-II (T2DM) diabetic status for a subject, the system comprising a processor and a memory in communication with the processor, the processor configured to provide the methods herein.

In a sixth aspect, there is provided a device for generating a diabetic status model for predicting a type-II (T2DM) diabetic status for a subject, the device comprising a processor and a memory in communication with the processor, the processor configured to provide the methods herein.

### DRAWINGS

A preferred embodiment of the present invention will now be described in detail with reference to the diagrams, in which:

FIG. 1 shows a system diagram in accordance with one or more embodiments.

FIG. 2 shows another system diagram in accordance with one or more embodiments.

FIG. 3 shows a device diagram in accordance with one or more embodiments.

FIG. 4 shows another device diagram in accordance with one or more embodiments.

FIGS. 5A, 5B, 5C, 5D, 5E, 5F, 5G, 5H, 5I and 5J show user interface diagrams in accordance with one or more embodiments.

FIG. 6 shows a computer-implemented method diagram in accordance with one or more embodiments.

FIG. 7 shows a computer-implemented method diagram in accordance with one or more embodiments.

FIG. 8 shows female model training results using individual recordings in Example 1. Left column shows results from Logistic Regression model, center column indicates results from Gaussian Naïve Bayes model, right column shows results from Support Vector Machine model. 1-feature models use only stdevF0; 2-feature models use stdevF0 and meanF0; 3-feature models use stdevF0, meanF0, and rapJitter; and 4-feature models use stdevF0, meanF0, rapJitter, and apq3Shimmer.

FIG. 9 shows male model training results using individual recordings in Example 1. Left column shows results from Logistic Regression model, center column indicates results from Gaussian Naïve Bayes model, right column shows results from Support Vector Machine model. 1-feature models use only meanInten; 2-feature models use meanInten and apq11Shimmer; 3-feature models use meanInten, apq11Shimmer, and stdevInten; and 4-feature models use meanInten, apq11Shimmer, stdevInten, and ppq5Jitter.

FIG. 10 shows a number of daily voice recordings per participants. d: day.

FIG. 11 shows the accuracy of the model using different number of voice recordings. The lines present the average accuracy for men (blue) and women (red). The shaded area shows the confidence interval. The numbers in the figure show the number of participants whose data were included in the analysis per each day.

FIG. 12 shows the accuracy of the model using 6 voice recordings per day. d: Day.

FIG. 13 shows the accuracy of the model using total 6 voice recordings from day 1 and day 2. D1: day 1, D2: day 2, N: number of samples from the day.

### DESCRIPTION OF VARIOUS EMBODIMENTS

Various apparatuses or methods will be described below to provide an example of the claimed subject matter. No example described below limits any claimed subject matter and any claimed subject matter may cover methods or apparatuses that differ from those described below. The claimed subject matter is not limited to apparatuses or methods having all of the features of any one apparatus or methods described below or to features common to multiple or all of the apparatuses or methods described below. It is possible that an apparatus or methods described below is not an example that is recited in any claimed subject matter. Any subject matter disclosed in an apparatus or methods described below that is not claimed in this document may be the subject matter of another protective instrument, for example, a continuing patent application, and the applicants, inventors or owners do not intend to abandon, disclaim or dedicate to the public any such invention by its disclosure in this document.

Furthermore, it will be appreciated that for simplicity and clarity of illustration, where considered appropriate, reference numerals may be repeated among the figures to indicate corresponding or analogous elements. In addition, numerous specific details are set forth in order to provide a thorough understanding of the examples described herein. However, it will be understood by those of ordinary skill in the art that the examples described herein may be practiced without these specific details. In other instances, well-known methods, procedures and components have not been described in detail so as not to obscure the examples described herein. Also, the description is not to be considered as limiting the scope of the examples described herein.

It should also be noted that the terms “coupled” or “coupling” as used herein can have several different meanings depending on the context in which these terms are used. For example, the terms “coupled” or “coupling” can have a mechanical, electrical or communicative connotation. For example, as used herein, the terms “coupled” or “coupling” can indicate that two elements or devices can be directly connected to one another or connected to one another through one or more intermediate elements or devices via an electrical element, electrical signal or a mechanical element depending on the particular context. Furthermore, the term “communicative coupling” indicates that an element or device can electrically, optically, or wirelessly send data to another element or device as well as receive data from another element or device.

It should also be noted that, as used herein, the wording “and/or” is intended to represent an inclusive-or. That is, “X and/or Y” is intended to mean X or Y or both, for example. As a further example, “X, Y, and/or Z” is intended to mean X or Y or Z or any combination thereof.

It should be noted that terms of degree such as “substantially”, “about” and “approximately” as used herein mean a reasonable amount of deviation of the modified term such that the end result is not significantly changed. These terms of degree may also be construed as including a deviation of the modified term if this deviation would not negate the meaning of the term it modifies.

Furthermore, the recitation of numerical ranges by endpoints herein includes all numbers and fractions subsumed within that range (e.g. 1 to 5 includes 1, 1.5, 2, 2.75, 3, 3.90, 4, and 5). It is also to be understood that all numbers and fractions thereof are presumed to be modified by the term “about” which means a variation of up to a certain amount of the number to which reference is being made if the end result is not significantly changed.

Some elements herein may be identified by a part number, which is composed of a base number followed by an alphabetical or subscript-numerical suffix (e.g. 112a, or 112₁). Multiple elements herein may be identified by part numbers that share a base number in common and that differ by their suffixes (e.g. 112₁, 112₂, and 112₃). All elements with a common base number may be referred to collectively or generically using the base number without a suffix (e.g. 112).

The example systems and methods described herein may be implemented in hardware or software, or a combination of both. In some cases, the examples described herein may be implemented, at least in part, by using one or more computer programs, executing on one or more programmable devices comprising at least one processing element, a data storage element (including volatile and non-volatile memory and/or storage elements), and at least one communication interface. These devices may also have at least one input device (e.g. a keyboard, a mouse, a touchscreen, and the like), and at least one output device (e.g. a display screen, a printer, a wireless radio, and the like) depending on the nature of the device. For example, and without limitation, the programmable devices (referred to below as computing devices) may be a server, network appliance, embedded device, computer expansion module, a personal computer, laptop, personal data assistant, cellular telephone, smart-phone device, tablet computer, a wireless device or any other computing device capable of being configured to carry out the methods described herein.

In some examples, the communication interface may be a network communication interface. In examples in which elements are combined, the communication interface may be a software communication interface, such as those for inter-process communication (IPC). In still other examples, there may be a combination of communication interfaces implemented as hardware, software, and a combination thereof.

Program code may be applied to input data to perform the functions described herein and to generate output information. The output information is applied to one or more output devices, in known fashion.

Each program may be implemented in a high-level procedural, declarative, functional or object-oriented programming and/or scripting language, or both, to communicate with a computer system. However, the programs may be implemented in assembly or machine language, if desired. In any case, the language may be a compiled or interpreted language. Each such computer program may be stored on a storage media or a device (e.g. ROM, magnetic disk, optical disc) readable by a general or special purpose programmable computer, for configuring and operating the computer when the storage media or device is read by the computer to perform the procedures described herein. Examples of the system may also be considered to be implemented as a non-transitory computer-readable storage medium, configured with a computer program, where the storage medium so configured causes a computer to operate in a specific and predefined manner to perform the functions described herein.

Furthermore, the example system, processes and methods are capable of being distributed in a computer program product comprising a computer readable medium that bears computer usable instructions for one or more processors. The medium may be provided in various forms, including one or more diskettes, compact disks, tapes, chips, wireline transmissions, satellite transmissions, internet transmission or downloads, magnetic and electronic storage media, digital and analog signals, and the like. The computer useable instructions may also be in various forms, including compiled and non-compiled code.

Various examples of systems, methods and computer programs products are described herein. Modifications and variations may be made to these examples without departing from the scope of the invention, which is limited only by the appended claims. Also, in the various user interfaces illustrated in the figures, it will be understood that the illustrated user interface text and controls are provided as examples only and are not meant to be limiting. Other suitable user interface elements may be used with alternative implementations of the systems and methods described herein.

As used herein, the term “user” refers to a user of a user device, and the term “subject” refers to a subject whose measurements are being collected. The user and the subject may be the same person, or they may be different persons in the case where one individual operates the user device and another individual is the subject. For example, in one embodiment the user may be a health care professional such as a nurse, doctor or dietitian and the subject is a human patient.

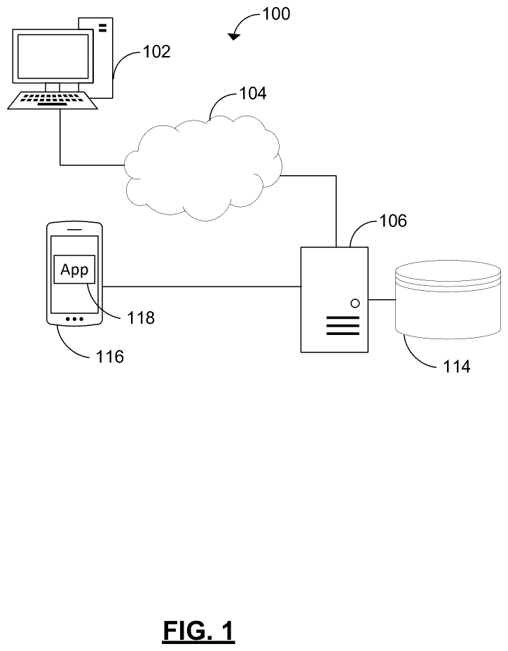

Reference is first made to FIG. 1, which shows a system diagram 100 of a type-2 diabetes mellitus (T2DM) status prediction system for determining a T2DM status prediction for a subject. The T2DM status prediction system includes one or more computer devices 102, a network 104, one or more servers 106, one or more data stores 114, and one or more user devices 116.

The one or more computer devices 102 may be used by a user such as a subject, an administrator, clinician, or other medical professional to access a software application (not shown) running on server 106 over network 104. In one embodiment, the one or more computer devices 102 may access a web application hosted at server 106 using a browser for reviewing T2DM status predictions given to the users using user devices 116. In an alternate embodiment, the one or more user devices 116 may download an application 118 (including downloading from an App Store such as the Apple® App Store or the Google® Play Store) for receiving T2DM status predictions.

The one or more user devices 116 may be any two-way communication device with capabilities to communicate with other devices. A user device 116 may be a mobile device such as mobile devices running the Google® Android® operating system or Apple® iOS® operating system. A user device 116 may be a smart speaker, such as an Amazon® Alexa® device, or a Google® Home® device. A user device 116 may be a smart watch such as the Apple® Watch, Samsung® Galaxy® watch, a Fitbit® device, or others as known. A user device 116 may be a passive sensor system attached to the body of, or on the clothing of, a user.

A user device 116 may be the personal device of a user, or may be a device provided by an employer. The one or more user devices 116 may be used by an end user to access the software application (not shown) running on server 106 over network 104. In one embodiment, the one or more user devices 116 may access a web application hosted at server 106 using a browser for determining T2DM status predictions. In an alternate embodiment, the one or more user devices 116 may download an application 118 (including downloading from an App Store such as the Apple® App Store or the Google® Play Store) for determining T2DM status predictions. The user device 116 may be a desktop computer, mobile device, or laptop computer. The user device 116 may be in communication with server 106, and may allow a user to review a user profile stored in a database at data store 114, including historical T2DM status predictions. The users using user devices 116 may provide one or more voice samples using a software application, and may receive a T2DM status prediction based on the one or more voice samples as described herein.

The application 118 may be an app for tracking health information, for example, the Fitbit® application, the Apple® Health® application, or the Google® Fit® application. The application 118 may be a nutrition or diet tracking application, for example, MyFitnessPal® or Noom®. The application 118 may be a dedicated application for T2DM prediction tracking. The application 118 may be a telehealth application that may provide remote interaction with one or more clinicians.

The one or more user devices 116 may each have one or more audio sensors. The one or more audio sensors may be in an array. The audio sensors may be used by a user of the software application 118 to record a voice sample into the memory of the user device 116. The one or more audio sensors may be an electret microphone onboard the user device, MEMS microphone onboard the user device, a Bluetooth enabled connection to a wireless microphone, a line in, etc.

The one or more user devices 116 may also include an additional caregiver device (not shown) or additional companion device (not shown). As described herein, caregiver and companion may be used interchangeably, and may refer to another individual separate from the subject/user of user device 116 who may be a friend, family member, caregiver, companion, or related individual to the subject/user. The caregiver may use the caregiver device (not shown) in order to monitor or be apprised of the alerts, notifications, and T2DM status predictions of the user 124. The caregiver device (not shown) may have a caregiver software application that may send a pairing request to the user device 116. The user may approve the pairing request, causing a pairing confirmation to be sent to the caregiver device. The pairing of the user device 116 and the caregiver device (not shown) may allow for alerts, notifications, and T2DM status predictions for the subject/user to be shared with a caregiver so that they may be informed of adverse situations.

The software application running on the one or more user devices 116 may communicate with server 106 using an Application Programming Interface (API) endpoint, and may send and receive voice sample data, user data, mobile device data, and mobile device metadata.

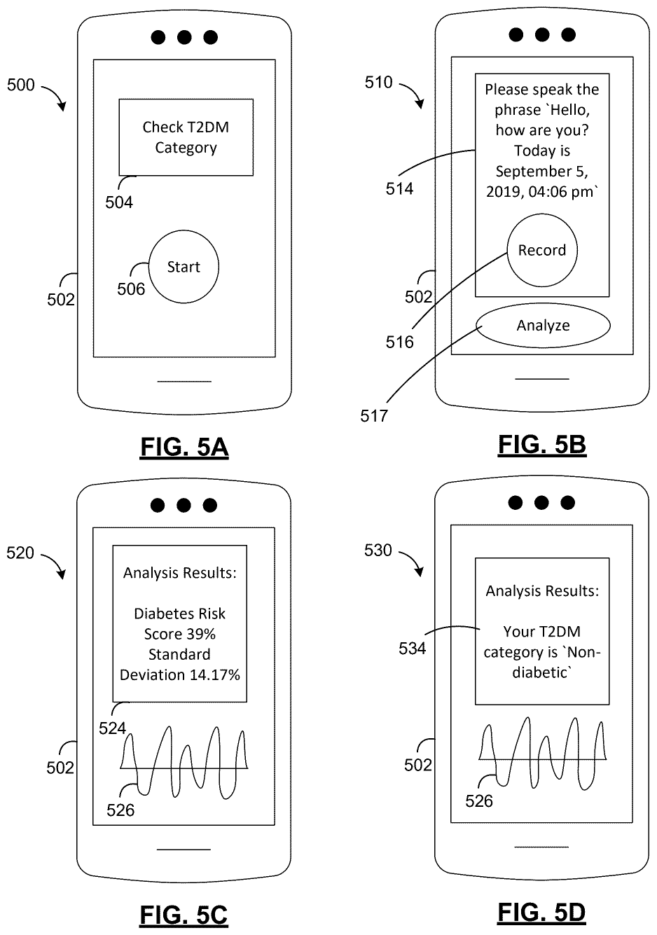

The software application running on the one or more user devices 116 may display one or more user interfaces on a display device of the user device, including, but not limited to, the user interfaces shown in FIGS. 5A, 5B, 5C, 5D, 5I and 5J.

A local wireless device of the one or more user devices 116 may allow for communication with one or more sensor devices 120. There may be one or more sensor devices.

The sensor device may be a wireless audio input device, such as a wireless microphone. The sensor device may transmit voice samples recorded proximate to the user to the user device 116, and may receive alarms or notifications from the user device 116 for presentation to the user. The sensor device may be worn on the body of user, on their clothing, or may be disposed proximate to the user.

Network 104 may be any network or network components capable of carrying data including the Internet, Ethernet, fiber optics, satellite, mobile, wireless (e.g. Wi-Fi, WiMAX), SS7 signaling network, fixed line, local area network (LAN), wide area network (WAN), a direct point-to-point connection, mobile data networks (e.g., Universal Mobile Telecommunications System (UMTS), 3GPP Long-Term Evolution Advanced (LTE Advanced), Worldwide Interoperability for Microwave Access (WiMAX), etc.) and others, including any combination of these.

The server 106 is in network communication with the one or more user devices 116 and the one or more computer devices 102. The server 106 may further be in communication with a database at data store 114. The database at data store 114 and the server 106 may be provided on the same server device, may be configured as virtual machines, or may be configured as containers. The server 106 and a database at data store114 may run on a cloud provider such as Amazon® Web Services (AWS®).

The server 106 may host a web application or an Application Programming Interface (API) endpoint that the one or more user devices 116 may interact with via network 104. The server 106 may make calls to the mobile device 116 to poll for voice sample data. Further, the server 106 may make calls to the database at data store 114 to query subject data, voice sample data, diabetic status model data, or other data received from the users of the one or more user devices 116. The requests made to the API endpoint of server 106 may be made in a variety of different formats, such as JavaScript Object Notation (JSON) or extensible Markup Language (XML). The voice sample data may be transmitted between the server 106 and the user device 116 in a variety of different formats, including MP3, MP4, AAC, WAV, Ogg Vorbis, FLAC, or other audio data formats as known. The voice sample data may be stored as Pulse-Code Modulation (PCM) data. The voice sample data may be recorded at 22,050 Hz or 44,100 Hz. The voice sample date may be collected as a mono signal, or a stereo signal. The voice sample data received by the data store 114 from the one or more user devices 116 may be stored in the database at data store 114, or may be stored in a file system at data store 114. The file system may be a redundant storage device at the data store 114, or may be another service such as Amazon® S3, or Dropbox.

The database of data store 114 may store subject information including T2DM status data, subject and/or user information including subject and/or user profile information, and configuration information. The database of data store 114 may be a Structured Query Language (SQL) such as PostgreSQL or MySQL or a not only SQL (NoSQL) database such as MongoDB.

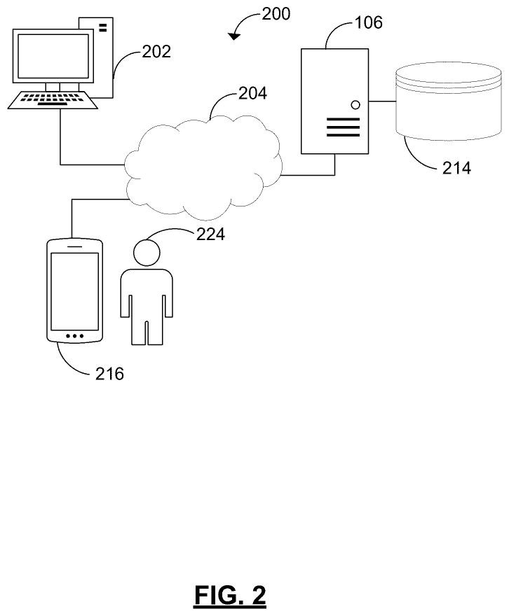

Referring next to FIG. 2 there is shown another system diagram 200 of an alternate embodiment of the T2DM prediction system for model generation. The one or more computer devices 202, the network 204, the one or more user devices 216, the server 206, and the data store 214 generally correspond to the one or more computer devices 102, the network 104, the one or more user devices 116, the server 106, and the data store 114 respectively of FIG. 1.

The system diagram 200 shows a data collection and model training embodiment, whereby audio data is collected from the audio input device or audio sensor device of user device 216. The collection of the audio data may be supplemented by metadata collection at the user device 216.

The user device 216 may run a software application configured to record a voice sample of the user 224 speaking. The audio recording may be supplemented by questionnaire data or the users medical record data including their T2DM status. In an alternate embodiment, clinical information may be provided to server 106 directly, including the subject's medical record data. For example, a clinician may provide medical record data to the server 106 directly.

The software application running on the one or more user devices 216 may communicate with server 106 using an Application Programming Interface (API) endpoint, and may send and receive voice sample data, user data, mobile device data, and mobile device metadata.

The software application running on the one or more user devices 216 may display one or more user interfaces to the user 224 who may be using user device 216, including those shown in FIGS. 5E, 5F, 5G and 5H. The software application running on the one or more user devices 216 may prompt the user to speak a particular prompt, and record a voice sample. The prompt may be a fixed sentence or utterance, or it may be a varied sentence or utterance. The software application may prompt the user 224 to provide a voice sample at particular times of day. For example, the software application may prompt user 224 to provide one or more voice samples in the afternoon.

The software application running on the one or more user devices 216 may communicate with server 106 by using requests made to the API endpoint of server 106 made in a variety of different formats, such as JavaScript Object Notation (JSON) or extensible Markup Language (XML). The voice sample data may be transmitted between the server 106 and the user device 216 in a variety of different formats, including MP3, MP4, AAC, WAV, Ogg Vorbis, FLAC, or other audio data formats as known. The voice sample data may be stored as Pulse-Code Modulation (PCM) data. The voice sample data may be recorded at 22,050 Hz or 44,100 Hz. The voice sample date may be collected as a mono signal, or a stereo signal. The voice sample data received by the data store 214 from the one or more user devices 216 may be stored in the database at data store 214, or may be stored in a file system at data store 214. The file system may be a redundant storage device at the data store 214, or may be another service such as Amazon® S3, or Dropbox.

The server 106, in addition to the data store 214 may further provide methods and functionality as described herein for generating a T2DM status prediction model.

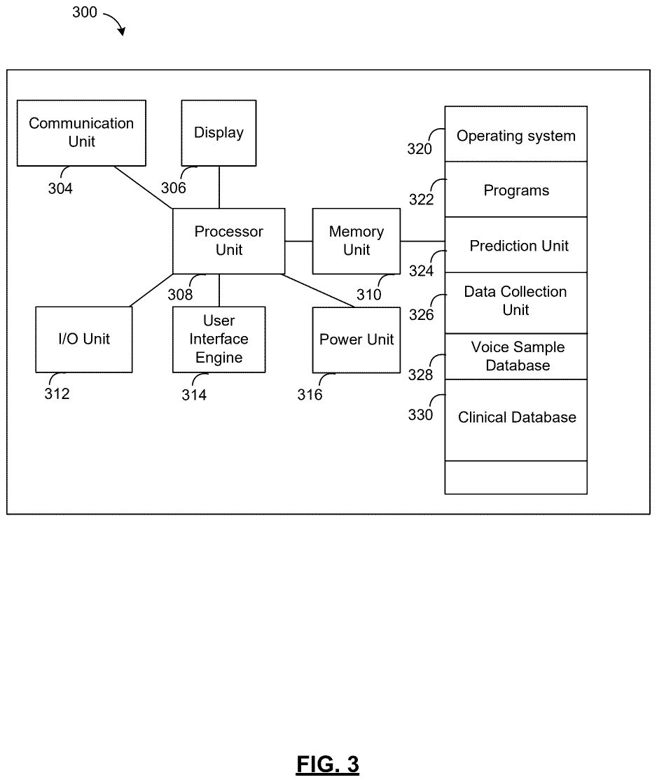

FIG. 3 shows a user device diagram 300 showing detail of the one or more user devices 116 in FIG. 1, and 216 in FIG. 2.

The user device 400 includes one or more of a communication unit 304, a display 306, a processor unit 308, a memory unit 310, I/O unit 312, a user interface engine 314, a power unit 316. The user device 300 may be a laptop, gaming system, smart speaker device, mobile phone device, smart watch or others as are known. The user device 300 may be a passive sensor system proximate to the user, for example, a device worn on user, or on the clothing of the user.

The communication unit 304 can include wired or wireless connection capabilities. The communication unit 304 can include a radio that communicates utilizing CDMA, GSM, GPRS or Bluetooth protocol according to standards such as IEEE 802.11a, 802.11b, 802.11g, or 802.11n. The communication unit 304 can be used by the mobile device 400 to communicate with other devices or computers.

Communication unit 304 may communicate with the wireless transceiver 318 to transmit and receive information via local wireless network. The communication unit 304 may provide communications over the local wireless network using a protocol such as Bluetooth (BT) or Bluetooth Low Energy (BLE).

The display 306 may be an LED or LCD based display, and may be a touch sensitive user input device that supports gestures.

The processor unit 308 controls the operation of the mobile device 300. The processor unit 308 can be any suitable processor, controller or digital signal processor that can provide sufficient processing power depending on the configuration, purposes and requirements of the user device 300 as is known by those skilled in the art. For example, the processor unit 308 may be a high performance general processor. In alternative embodiments, the processor unit 308 can include more than one processor with each processor being configured to perform different dedicated tasks. In alternative embodiments, it may be possible to use specialized hardware to provide some of the functions provided by the processor unit 308. For example, the processor unit 308 may include a standard processor, such as an Intel® processor, an ARM® processor or a microcontroller.

The processor unit 308 can also execute a user interface (UI) engine 314 that is used to generate various UIs, some examples of which are shown and described herein, such as interfaces shown in FIGS. 5A-5J.

The present systems, devices and methods may provide an improvement in the operation of the processor unit 308 by ensuring the analysis of voice data is performed using relevant biomarkers. The reduced processing required for the relevant biomarkers in the analysis (as compared with processing the superset of all biomarkers) reduces the processing burden required to make T2DM status predictions based on voice data.

The memory unit 310 comprises software code for implementing an operating system 320, programs 322, prediction unit 324, data collection unit 326, voice sample database 328, and clinical database 330.

The present systems and methods may provide an improvement in the operation of the memory unit 310 by ensuring the analysis of voice data is performed using relevant biomarkers and thus only relevant biomarker data is stored. The reduced storage required for the relevant biomarkers in the analysis (as compared with processing the superset of all biomarkers) reduces the memory overhead required to make T2DM status predictions based on voice data.

The memory unit 310 can include RAM, ROM, one or more hard drives, one or more flash drives or some other suitable data storage elements such as disk drives, etc. The memory unit 310 is used to store an operating system 320 and programs 322 as is commonly known by those skilled in the art.

The I/O unit 312 can include at least one of a mouse, a keyboard, a touch screen, a thumbwheel, a track-pad, a track-ball, a card-reader, an audio source, a microphone, voice recognition software and the like again depending on the particular implementation of the user device 300. In some cases, some of these components can be integrated with one another.

The user interface engine 314 is configured to generate interfaces for users to configure voice measurement, record training voice and glucose data, view T2DM clinical data, view voice sample data, view T2DM status predictions, etc. The various interfaces generated by the user interface engine 314 are displayed to the user on display 306.

The power unit 316 can be any suitable power source that provides power to the user device 300 such as a power adaptor or a rechargeable battery pack depending on the implementation of the user device 300 as is known by those skilled in the art.

The operating system 320 may provide various basic operational processes for the user device 300. For example, the operating system 320 may be a mobile operating system such as Google® Android® operating system, or Apple® iOS® operating system, or another operating system.

The programs 322 include various user programs so that a user can interact with the user device 300 to perform various functions such as, but not limited to, viewing T2DM data, voice data, recording voice samples, receiving and viewing, receiving any other data related to T2DM status predictions, as well as receiving messages, notifications and alarms as the case may be. The programs 322 may include a telephone calling application, a voice conferencing application, social media applications, and other applications as known. The programs 322 may include the Fitbit® application, the Apple® Health® application, or the Google® Fit® application. The programs 322 may include a nutrition or diet tracking application, for example, MyFitnessPal® or Noom®. The programs 322 may include a dedicated application for T2DM prediction tracking. The programs 322 may include a telehealth application that may provide remote interaction with one or more clinicians. The programs 322 may make calls, requests, or queries to the prediction unit 324, the data collection unit 326, the voice sample database 328, and the clinical database 330. The programs 322 may be downloaded from an application store (“app store”) such as the Apple® App Store® or the Google® Play Store®.

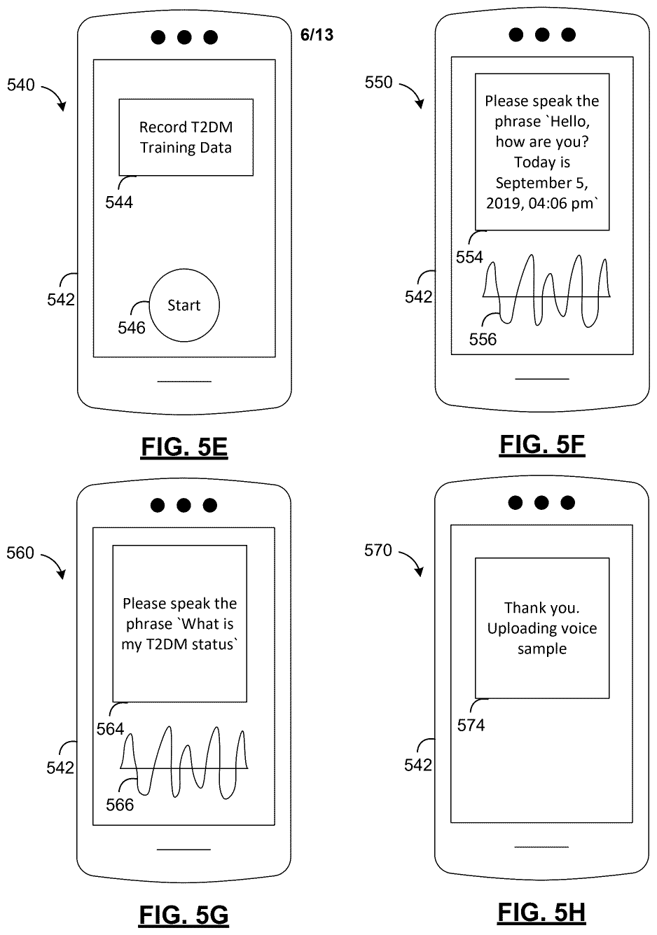

In one or more embodiments, the programs 322 may include a diabetic management application. The diabetic management application may record voice samples from the user and report the user's T2DM status predictions. Such a fitness application may integrate with a health tracker of the individual such as a Fitbit®, or Apple® Watch such that additional exercise, or measurement data may be collected. The diabetic management application may record historical T2DM status predictions in order to determine changes in the user's T2DM status predictions. The embodiments described herein may allow for a diabetic user, a pre-diabetic user, and a non-diabetic user to check T2DM status predictions using voice samples. The diabetic management application may use the T2DM status predictions to generate a notification to a user. The notification may include a mobile notification such as an app notification, a text notification, an email notification, or another notification that is known. The diabetic management application may operate using the method of FIG. 6.

In one or more embodiments, the programs 322 may include a smart speaker application, operable to interact with a user using voice prompts, and receptive of voice commands. In such an embodiment, the voice commands the user provides as input may be used as voice sample data as described herein. In this case, a user may request their T2DM status prediction by prompting the smart speaker “Alexa, what is my type-2 diabetic status prediction?” or similar. The smart speaker application may passively monitor the user's T2DM status by way of the voice command voice samples, and may alert the user if it changes. The smart speaker application may follow the method of FIG. 6.

In one or more embodiments, the programs 322 may include a smart watch application for outputting information including a T2DM status prediction on a watch face. The smart watch application may enable a user to provide voice prompts using an input device of the watch and check T2DM status on an output device of the watch. The smart watch application may follow the method of FIG. 6.

In one or more embodiments, the programs 322 may include a nutrition application which may determine a diet recommendation for a user based on their T2DM status prediction. The nutrition application may also recommend food intake or diet changes to the user. The nutrition application may follow the method of FIG. 6.

In one or more embodiments, the programs 422 may include a pre-diabetic lifestyle application that may track the user's T2DM status predictions, and may output predictions of disease susceptibility. The pre-diabetic lifestyle application may provide lifestyle change recommendations to a pre-diabetic user. The pre-diabetic lifestyle application may follow the method of FIG. 6.

The lifestyle application may allow for the user to select lifestyle criteria and lifestyle values. The lifestyle criteria may correspond to items such as “tobacco usage”, “alcohol intake”, “exercise level” or other such behavior and lifestyle descriptors that may be associated with an increased risk of type-II diabetes. Each lifestyle criteria may correspond to a lifestyle value. For example, a “tobacco intake” may select 5 cigarettes per day as the corresponding lifestyle value. The lifestyle values may similarly correlate to number of units of alcohol per day, number of minutes of exercise per day, number of steps per day, volume of water consumer per day, etc.

The lifestyle criteria may be diarized in a lifestyle request. The lifestyle request may allow a user to document at different times, lifestyle changes which may have an impact upon their type-II diabetes risk.

Based on the T2DM status prediction, and the user's diarized lifestyle requests, the lifestyle application may determine (or may request from a server) a lifestyle change recommendation.

In one or more embodiments, the programs 322 may include a video conferencing application. The video conferencing application may follow the method of FIG. 6.

In one or more embodiments, the programs 322 may include a pre-diabetic screening application. The pre-diabetic screening application may assist a medical professional or another user to provide pre-diabetic screening to determine a diabetic risk profile based on a T2DM status predictions. The pre-diabetic screening application may be combined and integrated with a validated prediabetes screener (e.g. CANRISK), and may include a questionnaire in addition to a voice sample analysis. For example, the pre-diabetic screening application may incorporate at least one screening question that provide information related to risk factors for pre-diabetes or diabetes such as body mass index (BMI), weight, blood pressure, disease comorbidity, family history, age, race or ethnicity and physical activity. The at least one screening question may be used as feature inputs and combined with the voice features in the predictive model. The pre-diabetic screening application may be used by a medical professional or may be provided directly to a user. The pre-diabetic screening application may follow the method of FIG. 6.

In one or more embodiments, the programs 322 may include a passive T2DM monitoring application that may receive audio inputs, transmit voice samples to a server, optionally receive T2DM status predictions, and optionally provide alerts to the user's device to the user automatically and without user prompting. In one or more embodiments, the passive sensor application may be connected wirelessly to a user device such as a mobile phone, and may cause an email, text message, or application notification to be displayed to a user on the user device. The passive sensor application may follow the method of FIG. 6.

In one or more embodiments, the passive sensor application may provide a notification to the user such as to take medication (e.g. insulin), consume or avoid certain foods or otherwise follow a therapeutic plan. The passive sensor application may follow the method of FIG. 6.

In one or more embodiments, the programs 322 may include an educational application. For example, in one embodiment programs 322 include an educational application for helping subjects understand their T2DM status. The educational program may communicate recommended diet and behavioral changes to the user, and may use the user's voice samples to tailor educational content presented to them on the user device. The educational application may follow the method of FIG. 6.

In one or more embodiments, the programs 322 may include a subject tracker for a plurality of subjects. The subject tracker may provide a user interface providing information and T2DM status predictions generated periodically from the subjects. The T2DM status predictions may be provided to the medical professional in order to e.g. collect clinical trial data or adjust a treatment plan for a subject in the plurality of subjects. The user interface may include a reporting interface for the plurality of subjects, or alternatively may provide email, text message, or application notifications to the medical professional about one or more subjects based on subject T2DM status predictions, disease susceptibility, or other predicted subject data. The subject tracker may follow the method of FIG. 6.

In one or more embodiments, the programs 322 may include a caregiver application for friends and family members of type-II diabetic subjects. The user of the caregiver application may receive T2DM status predictions for another subject. The caregiver application may be paired with a user profile of a user of one of the T2DM status prediction programs described herein. The pairing may provide a caregiver of a subject with type-II diabetes alerts or notifications based on voice samples of the subject so that they are aware of adverse situations and allow them to intervene to correct them if required. The subject paired with the caregiver may record their voice samples using a passive sensor device attached to their body, and/or clothing. The caregiver application may follow the method of FIG. 6.

The prediction unit 324 receives voice data from the audio source connected to I/O unit 312 via the data collection unit 326, and may transmit the voice data to the server (see e.g. 106 in FIGS. 1 and 2). In response, the server may operate the method as described in FIG. 6 to generate a T2DM status prediction for the subject, and may respond with the T2DM status prediction to the user device. The voice sample data may be stored in the voice sample database 328 along with the prediction data. Prediction unit 324 may determine predictive messages based on the voice model and the voice sample data. The predictive messages may be displayed to a user of the mobile device 300 using display 306. The predictive messages may include the T2DM status prediction.

In an alternate embodiment, the prediction unit 324 of the mobile device 300 may include a T2DM status prediction model, and may operate the method as described in FIG. 6 to generate a T2DM status prediction for the subject on the mobile device itself. In this alternate embodiment, the voice sample data may be stored in the voice sample database 328 along with the prediction data.

The data collection unit 326 receives voice sample data from an audio source connected to the I/O unit 312.

In one or more embodiments, the data collection unit 326 receives medical record data, self-reported questionnaire data, or other clinical data and may store it in the clinical database 330. The data collection unit 326 may receive the clinical data and may transmit it to a server. The data collection unit 326 may supplement the clinical data that is received from the user of the device 300 with mobile device data and mobile device metadata.

The voice sample database 328 may be a database for storing voice samples received by the user device 300. The voice sample database 328 may receive the data from the data collection unit 326.

The clinical database 330 may be a database for storing medical record data, self-reported questionnaire data, or other clinical data. The measurement database 430 may receive the data from the data collection unit 426.

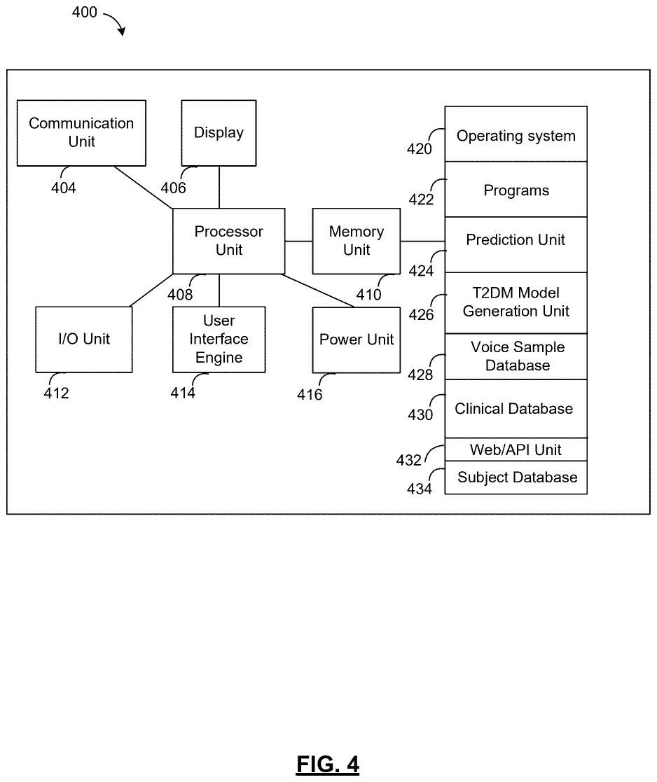

FIG. 4 shows a server diagram showing detail of the server 106 in FIGS. 1 and 2. The server 400 includes one or more of a communication unit 404, a display 406, a processor unit 408, a memory unit 410, I/O unit 412, a user interface engine 414, and a power unit 416.

The communication unit 404 can include wired or wireless connection capabilities. The communication unit 404 can include a radio that communicates using standards such as IEEE 802.11a, 802.11b, 802.11g, or 802.11n. The communication unit 404 can be used by the server 400 to communicate with other devices or computers.

Communication unit 404 may communicate with a network, such as network 104 and 204 (see FIGS. 1 and 2 respectively).

The display 406 may be an LED or LCD based display, and may be a touch sensitive user input device that supports gestures.

The processor unit 408 controls the operation of the server 400. The processor unit 408 can be any suitable processor, controller or digital signal processor that can provide sufficient processing power depending on the configuration, purposes and requirements of the server 400 as is known by those skilled in the art. For example, the processor unit 408 may be a high performance general processor. In alternative embodiments, the processor unit 408 can include more than one processor with each processor being configured to perform different dedicated tasks. The processor unit 408 may include a standard processor, such as an Intel® processor or an AMD® processor.

The processor unit 408 can also execute a user interface (UI) engine 414 that is used to generate various UIs for delivery via a web application provided by the Web/API Unit 430, some examples of which are shown and described herein, such as interfaces shown in FIG. 5A-5J.

The memory unit 410 comprises software code for implementing an operating system 420, programs 422, prediction unit 424, T2DM model generation unit 426, voice sample database 428, clinical database 430, Web/API Unit 432, and subject database 434.

The memory unit 410 can include RAM, ROM, one or more hard drives, one or more flash drives or some other suitable data storage elements such as disk drives, etc. The memory unit 410 is used to store an operating system 420 and programs 422 as is commonly known by those skilled in the art.

The I/O unit 412 can include at least one of a mouse, a keyboard, a touch screen, a thumbwheel, a track-pad, a track-ball, a card-reader, an audio source, a microphone, voice recognition software and the like again depending on the particular implementation of the server 400. In some cases, some of these components can be integrated with one another.

The user interface engine 414 is configured to generate interfaces for users to configure voice measurement, record training voice data, view T2DM prediction data, view voice sample data, view T2DM prediction, etc. The various interfaces generated by the user interface engine 414 may be transmitted to a user device by virtue of the Web/API Unit 432 and the communication unit 404.

The power unit 416 can be any suitable power source that provides power to the server 400 such as a power adaptor or a rechargeable battery pack depending on the implementation of the server 400 as is known by those skilled in the art.

The operating system 420 may provide various basic operational processes for the server 400. For example, the operating system 420 may be a server operating system such as Ubuntu® Linux, Microsoft® Windows Server® operating system, or another operating system.

The programs 422 include various user programs. They may include several hosted applications delivering services to users over the network, for example, a voice conferencing server application, a social media application, and other applications as known.

In one or more embodiments, the programs 422 may provide a public health platform that is web-based, or client-server based application via Web/API Unit 432 that provides for health research on a large population of subjects. The health platform may provide population health researchers the ability to conduct large N surveillance studies to map the incidence and prevalence of diabetes and prediabetes. The public health platform may provide access for queries and data analysis of the voice sample database 428, the clinical database 430, and the subject database 434. The health platform may allow for population health research on different groups, including based on demographic information, the subject's non-diabetic, diabetic or pre-diabetic status.

In one or more embodiments, the programs 422 may provide a public health platform that is web-based, or client server based via a Web/API Unit 432 that provides type-II diabetic risk stratification for a population of subjects. This may include a patient population of a medical professional who is a user of the public health platform. For example, the medical professional may be able to receive a 24 h view into T2DM predictions for their patients to further identify the subject's risk levels.

The prediction unit 424 receives voice data from a user device over a network at Web/API Unit 432, and may operate the method as described in FIG. 6 to generate a T2DM status prediction for the subject. The server may respond with the T2DM status prediction to the user device via a message from the Web/API Unit 432. The voice sample data may be stored in the voice sample database 428 along with the prediction data. Prediction unit 424 may determine predictive messages based on the T2DM status model and the voice sample data.

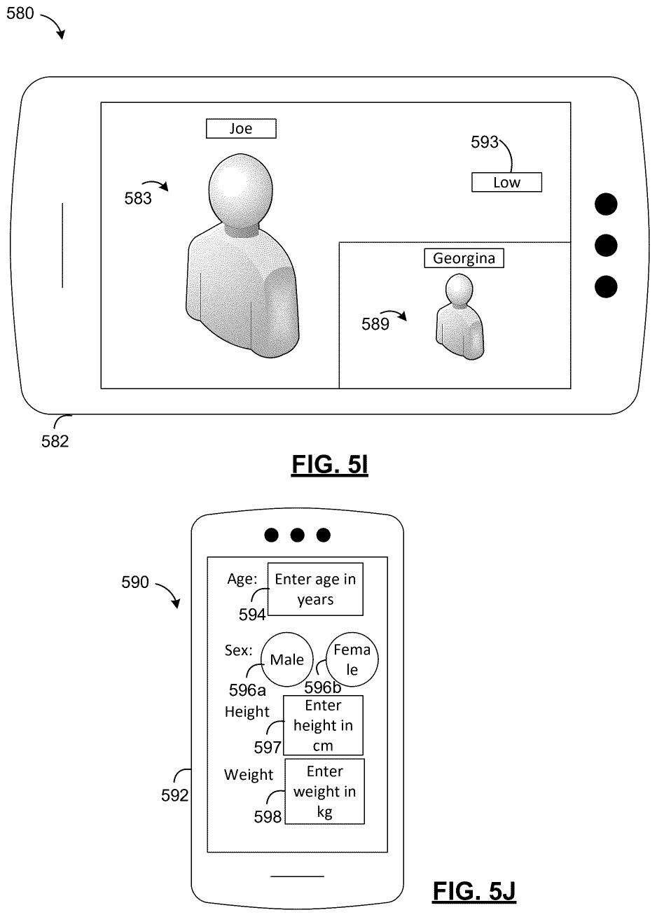

The T2DM model generation unit 426 receives voice data from voice sample database 428, clinical data from clinical database 430, and subject information from subject database 434. The T2DM model generation unit 426 may generate a T2DM status prediction model based on the method of FIG. 7.

The voice sample database 428 may be a database for storing voice samples received from the one or more user devices via Web/API Unit 532. The voice sample database 428 may include voice samples from a broad population of subjects interacting with user devices. The voice samples in voice sample database 428 may be referenced by a subject identifier that corresponds to an entry in the subject database 434 or the clinical database 43-. The voice sample database 428 may include voice samples for a population of subjects, including more than 10,000, more than 100,000 or more than a million subjects. The voice sample database 428 may include voice samples from many different audio sources, including passive sensor devices, user devices, PBX devices, smart speakers, smart watches, game systems, voice conferencing applications, etc.

The clinical database 430 may be a database for storing medical record data, questionnaire data, or other clinical data received from the one or more user devices via Web/API Unit 532. The clinical database 430 may include blood glucose measurements from a broad training population of subjects who have performed the training actions using the one or more user devices. The blood glucose measurements in clinical database 430 may be referenced by a subject identifier that corresponds to an entry in the subject database 434. The glucose measurement database 430 may include glucose measurements corresponding to voice samples for a population of subjects, including more than 1,000, more than 10,000 or more than 100,000 subjects.

The Web/API Unit 432 may be a web based application or Application Programming Interface (API) such as a REST (REpresentational State Transfer) API. The API may communicate in a format such as XML, JSON, or other interchange format.

The Web/API Unit 432 may receive a T2DM status prediction request including a voice sample, may apply methods herein to determine a T2DM status prediction, and then may provide the prediction in a T2DM status prediction response. The voice sample, values determined from the voice sample, and other metadata about the voice sample may be stored after receipt of a T2DM status prediction request in voice sample database 428. The predicted T2DM status may be associated with the voice sample database entry, and stored in the subject database 434.

The Web/API Unit 432 may receive a training request, including clinical data and voice samples. The voice samples, values determined from the voice samples, and other metadata about the voice samples may be stored after receipt of a T2DM status prediction request in voice sample database 428. The corresponding clinical data may be associated with the voice sample entry in the voice sample database 428 and stored in the clinical database 430.

The Web/API Unit 432 may receive a nutritional recommendation request including a voice sample, may apply methods herein to determine a T2DM status prediction and a nutritional recommendation, and then may provide the T2DM status prediction and the nutritional recommendation in a response. The nutrition recommendation may use coarse T2DM status predictions to recommend nutrients to the user so that the user can adjust their diet. The voice sample of the nutritional recommendation request may be stored in voice sample database 428. The nutritional recommendation provided in response may be associated with the voice sample entry in voice sample database 428 and stored in the subject database 434.

The Web/API Unit 432 may receive a food check request including a food identifier and a voice sample. The Web/API Unit 432 may determine whether it's acceptable for the user to consume the food identified by the food identifier based on their current T2DM status as predicted based on the voice sample. The Web/API Unit 432 may make a call to a third party database, such as a food or nutrition database, in order to determine nutritional values of the food identified by the food identifier. In response to the food check request, the Web/API Unit 432 may reply with a food check response including an indication of whether it is acceptable for the user/subject to consume the food. The food check response may include an unlock command which may be used by the user device to unlock a corresponding food container. The voice sample of the food check may be stored in voice sample database 428. The food identifier may be associated with the voice sample entry in voice sample database 428 and stored in subject database 434. The food check response, including whether the subject is permitted to consume the food, may be associated with the food identifier, the voice sample entry in the voice sample database 428, and stored in subject database 434.

The Web/API Unit 432 may receive a lifestyle journaling request including one or more lifestyle criteria and a corresponding one or more lifestyle values. The lifestyle criteria may include a criteria of the user, such as weight, blood pressure, caloric intake, tobacco smoking intake, alcohol intake, illicit substance intake, pharmaceutical intake, or other criteria as are known. Optionally, each lifestyle criteria may be provided with a lifestyle value. For example, for “alcohol intake”, a user may indicate “3 drinks per week”. The lifestyle journaling request may be made by a user device and may include a voice sample or other data based on the sample such as a blood glucose level. The voice sample may be stored in voice sample database 428. The one or more lifestyle criteria and the corresponding one or more lifestyle values may be associated with the voice sample or other data and may be stored in subject database 434. In response to the lifestyle journaling request, a lifestyle response may be transmitted to the user device. The response may include a T2DM status trend indication, a disease progression score, or a relative value. The trend or progression scores may be determined based upon the user/subject's historical lifestyle criteria/values. For example, if a user decreases their alcohol intake from “5 drinks per week” to “3 drinks per week”, the lifestyle response may include a trend or indication of the user's decreased susceptibility to type-II diabetes. Optionally, the lifestyle response may include an indicator or flag that the user's medication or therapeutic plan should be reviewed or changed with a health professional.

The Web/API Unit 432 may receive a screening question request from a user device. In response, the Web/API Unit 432 may send at least one pre-diabetic screening questions to the user device.

The Web/API Unit 432 may receive a screening answer request, including a voice sample and at least one answer to a corresponding at least one pre-diabetic screening questions. The Web/API Unit 432 may determine a pre-diabetic risk profile based on the voice sample and the one or more answers, and may transmit it in response to the user device in a pre-diabetic screening response including the risk profile. In one embodiment, the at least one screening answer comprise clinicopathological information such as, but not limited to, information on one or more of height, weight, BMI, diabetes status, blood pressure, disease comorbidity, family history, age, race or ethnicity and physical activity.

The subject database 434 may be a database for storing subject information, including one or more clinicopathological values about each subject. Further, the subject database 434 may include the subject's food checks, references to the subject's voice sample entries in the voice sample database 428, food identifiers used in food check requests, nutritional recommendation requests, and nutritional recommendation responses. Each subject may have a unique identifier, and the unique identifier may reference voice samples in the voice sample database 428 and clinical data in the clinical database 430. The subject database 434 may include subject information for a population of subjects, including more than 10,000, more than 100,000 or more than a million subjects. The subject database may have anonymized subject data, such that it does not personally identify the subjects themselves.

Referring next to FIGS. 5A, 5B, 5C, 5D and 5J together, there are example user interfaces 500, 510, 520, 530 respectively showing a subject collecting a voice sample and receiving a T2DM status prediction. The device displaying the user interfaces 500, 510, 520 and 520 may be a vocal analysis pre-screening smartphone application that analyzes vocal patterns to detect markers consistent with those found in Type 2 diabetes patients. The analysis may done remotely via an API to a server (e.g. server 106 in FIG. 1). The vocal analysis pre-screening smartphone application may generate a proprietary score (e.g. as shown in FIG. 5C or 5D), reporting risk factors based on the identified vocal biomarkers.

The score may be a representation of the probability that this person has diabetes, using a predictive model trained on age, sex, BMI and vocal features as shown in FIG. 5C or may alternatively be a classification as shown in FIG. 5D.

The vocal analysis pre-screening smartphone application may be designed for use by both the general public outside of clinical environments, enabling at-home risk assessment, and by healthcare professionals as a preliminary screening tool in clinical settings. It may also be provided to help individuals understand how their lifestyle choices influence their risk factors for type 2 diabetes. The vocal analysis pre-screening smartphone application may encourage users to consult their family physician or nurse practitioner based on the score obtained.

At interface 500, there is a user interface shown to a user at a user device 502 who desires to receive a T2DM status prediction. To initiate the prediction, the user is prompted to begin the T2DM status check by selecting a start button 506. Once start is selected, the audio input of the user device begins recording the voice sample into memory of the user device 502.

In an alternate embodiment, the user may receive a notification on the user device 502 to initiate the voice sampling, and by selecting the notification may be presented with interface 500 to initiate the collection. The notification to the user to initiate the voice sampling may be determined based on the time of day.

In response to the user selecting the start button 506, a variable prompt interface 510 is shown, prompting the user to read the prompt 514. The prompt may be a variable prompt 514 as shown in addition to a record button 516, and may change subject to subject, or for each voice sample that is recorded. During the voice sample collection, the user interface 510 may show a voice sample waveform (not shown) on the display. The user may select the analyze button 517 to access the prediction.

In an alternate embodiment, the user may also be prompted to provide information after selecting start 506 as described in FIG. 5J. For example, the user may be prompted to provide age 594, sex 596a or 596b (or, alternate options may be provided for non-binary individuals), height 597 and weight 598.

Alternatively, a static prompt to user interface 520 may instead be shown to a subject and the prompt 524 may be static. Each subject may speak the same prompt out loud for every voice sample. During the voice sample collection, the user interface 520 may show a voice sample waveform 526 on the display.

In response to completing the voice prompt (either static or variable) and submitting the analyze button 517, a T2DM diabetes risk score 524 or a T2DM status prediction 534 may be made in a T2DM status prediction interface (e.g. FIG. 5C or 5D).

The T2DM status prediction 524 may be a numerical prediction, i.e. a percentage risk and a standard deviation. The T2DM status prediction 534 may be a categorical prediction, i.e. ‘Low’, ‘Medium’, and ‘High’ or ‘non-diabetic’, ‘pre-diabetic’ and ‘diabetic’ or a quantitative confidence level i.e. 80% chance of T2DM diabetic. As described herein, the T2DM status prediction 534 may be for a plurality of categorical predictions, optionally categorical predictions that may appear continuous such as numerical values. The prediction may be generated by a server, or may be generated by the user device itself.

Referring next to 5E, 5F, 5G, and 5H together, there are example interfaces 540, 550, 560, and 570 respectively showing a subject performing training actions on a user device 542.

At interface 540, there is a user interface shown to a user at a user device 542 who desires to perform a training action. The interface 540 may provide a questionnaire to the user to self-identify their T2DM status based on their own knowledge. The subject may initiate the training action by selecting the start button 546.

In an alternate embodiment, the user may receive a notification on the user device 542 to initiate the training action, and by selecting the notification may be presented with interface 540 to initiate the training action. The notification to the user to perform the training action may be determined based on the time of day.

In response to the user selecting the start button 546, a variable training interface 550 may be displayed on the user device 542 providing a variable prompt 554 for the subject to read. A voice waveform indication 556 may be displayed to the user.

Alternatively, in response to the user selecting the start button 546, a static training interface 560 may be displayed to the user selecting the start button 546, providing a static prompt 564 for the subject to read. A voice waveform indication 566 may be displayed to the user.

In response to the user selecting the start button 546, a subject voice sample data may be recorded from an audio input of the user device 542 into memory.

In response to the user completing the voice sample data and the clinical data questionnaire collection, a completion interface 570 may be displayed indicating that the data is being uploaded to a server.

Referring next to FIG. 5I there is shown an example user interface 580 showing a video conferencing application including automatic T2DM status predictions.

The T2DM prediction software application may be integrated with an existing software application, such as a telehealth or videoconferencing application or a social network application in order to provide T2DM status prediction data automatically. In one example, the software application may be integrated with a video conferencing application such as Zoom®.

In the video conferencing interface 580, a user Joe 583 is speaking to clinician Georgina 589 are shown on the display of user device 582. Based on Joe's 583 voice samples transmitted using the video conferencing application, the methods herein may be used in order to provide a T2DM status prediction to his clinician Georgina 589. For example, Joe has a T2DM prediction of ‘Low’ 593 (or, in an alternate embodiment, the prediction may be “non-diabetic). As described herein, the T2DM status prediction may be one of ‘Low’, ‘Medium’, or ‘High’. Alternatively, the status categories may be “non-diabetic”, “pre-diabetic” and “diabetic”. Alternatively, the prediction may include a numerical confidence prediction related to T2DM status.

Referring next to FIG. 6, there is a computer-implemented method diagram 600 showing a computer-implemented method for generating a type-II (T2DM) diabetic status prediction for a subject.

At 602, a diabetic status prediction model is provided at a memory.

At 604, a voice sample is received, the voice sampled is received at a processor in communication with the memory.

At 606, at least one voice biomarker feature value from the voice sample for at least one predetermined voice biomarker feature is extracted at the processor.

At 608, the type-II (T2DM) diabetic status prediction for the subject is determined based on the at least one voice biomarker feature value and the diabetic status prediction model at the processor.

At 610, the type-II (T2DM) diabetic status prediction for the subject is output at an output device, or an output based on the diabetic status prediction.

In one or more embodiments, each of the at least one voice biomarker feature value may be selected from the group comprising: a statistical feature category, a shimmer feature category, and a jitter feature category.

In one or more embodiments, the statistical feature category may comprise a mean pitch feature value, a pitch standard deviation feature value, a mean intensity feature value, an intensity standard deviation feature value and a harmonic-to-noise ratio feature value; the shimmer feature category may comprise a localShimmer feature value, a localdbShimmer feature value, an apq3Shimmer feature value, an apq5Shimmer feature value, and an apq11Shimmer feature value; and the jitter feature category may comprise a localJitter feature value, a localabsJitter feature value, a rapJitter feature value and a ppq5Jitter feature value.

In one or more embodiments, the method may further include: preprocessing, at the processor, the voice sample by: storing, at a database in communication with the processor, a plurality of historical voice samples of the subject; and averaging the voice sample based on at least one of the plurality of historical voice samples of the subject.

In one or more embodiments, the voice sample may comprise a predetermined phrase vocalized by the subject; and the voice sample is received from a user device in network communication with the processor.

In one or more embodiments, the predetermined phrase may be displayed to the subject on a display device of the user device.

In one or more embodiments, the method may further include transmitting, to the user device in network communication with the processor, the type-II (T2DM) diabetic status prediction for the subject, wherein the outputting of the diabetic status prediction for the subject occurs at the user device.

In one or more embodiments, the diabetic status prediction may comprise a categorical prediction.

In one or more embodiments, the categorical prediction may be one selected from the group of: a type-II (T2DM) diabetic category, and a normal category.

In one or more embodiments, the determining the diabetic status prediction for the subject may be based on at least one selected from the group of: vocal parameter data of the subject, age data of the subject, and Body Mass Index (BMI) data of the subject.

In one or more embodiments, the diabetic status prediction model may comprise at least one selected from the group of a Logistic Regression (LR) model, a Naïve Bayes (NB) model, and a Support Vector Machine (SVM) model.

In one or more embodiments, the diabetic status prediction model may comprise an ensemble model. This may include, averaging all the prediction probabilities within an individual, averaging the voice prediction results with the T2DM prevalence at the participant's age, averaging the voice prediction results with the T2DM prevalence at the participant's BMI, and/or a combination of these methods.

Referring next to FIG. 7, there is shown a computer-implemented method diagram 700 for generating a diabetic status model for predicting a type-II (T2DM) diabetic status for a subject in accordance with one or more embodiments.

At 702, providing, at a memory: (704) a plurality of diabetic status labels, the plurality of diabetic status labels identifying a corresponding diabetic status for a plurality of training subjects; and (706) a plurality of voice samples collected from the plurality of training subjects at a plurality of time points, each of the voice samples in the plurality of voice samples associated with a corresponding diabetic status label in the plurality of diabetic status labels.

In one or more embodiments, due to a large proportion of non-diabetic participants that skewed younger than the T2DM sample, some non-diabetic recordings that have no age and BMI match in the T2DM arm may be excluded from the matched sample. An equal number of T2DM and non-diabetic participants may be included in the matched dataset such that 50% of T2DM males and 61% of T2DM females were included. Statistical analysis and prediction model training may be performed on the matched dataset, and the remaining data may be used to test the fully trained model (referred to as the “test dataset”).

At 708, determining, at the processor, a plurality of voice feature values for a corresponding plurality of voice features for each of the voice samples in the plurality of voice samples.

In one or more embodiments, the voice feature values may be extracted using Parselmouth, a publicly available Python integration for Praat, a voice and speech analysis software.

In one or more embodiments, the plurality of voice features can include a total of 14 voice features extracted from each audio recording including pitch, pitch standard deviation, intensity, intensity standard deviation, harmonic noise ratio, 5 features corresponding with shimmer, and 4 features corresponding with jitter.

In one or more embodiments, the plurality of voice features may selected from the listing of voice features in Appendix A.

In one or more embodiments, jitter and shimmer values may be selected to be evaluated in addition to the pitch, intensity and harmonic noise ratio vocal parameters.

In one or more embodiments, the features may be selected based on the Cohen's d effect size in the age matched data set. Cohen's d may be calculated based on Equation 1.

In one or more embodiments, the features may be added iteratively to the models based on decreasing absolute value of Cohen's d calculated from the matched dataset. Selected features may be added to the featureset if they provide significantly different between the non-diabetic group and the diabetic group in both the matched sample and the entire dataset.

At 710, generating at the processor, the diabetic status model based on the plurality of voice samples and the plurality of voice feature values.

In one or more embodiments, the diabetic status model may be generated based on 5-fold cross validation based on the matched dataset in order to select the model, featureset and threshold for prediction. After cross validation, the entire matched dataset may be used for model training. All recordings corresponding to individuals within the matched dataset may be used. The model performance may be assessed on the testing set (i.e. the data not used in the matched dataset) using the pretrained model, feature set and threshold determined through cross validation of the matched data.

In one or more embodiments, each of the at least one voice biomarker feature value may be selected from the group comprising: a statistical feature category, a shimmer feature category, and a jitter feature category.

In one or more embodiments, the statistical feature category may comprise a mean pitch feature value, a pitch standard deviation feature value, a mean intensity feature value, an intensity standard deviation feature value and a harmonic-to-noise ratio feature value; the shimmer feature category may comprise a localShimmer feature value, a localdbShimmer feature value, an apq3Shimmer feature value, an apq5Shimmer feature value, and an apq11Shimmer feature value; and the jitter feature category may comprise a localJitter feature value, a localabsJitter feature value, a rapJitter feature value and a ppq5Jitter feature value.

In one or more embodiments, the method may further include selecting, at the processor, a subset of voice feature values from the plurality of voice feature values for each voice sample based on a Cohen's d effect size of each voice feature value; and wherein the generating at the processor, the diabetic status model may be based on the plurality of voice samples and the subset of voice feature values.

In one or more embodiments, the method may further include preprocessing, at the processor, each voice sample by averaging the voice sample based on at least one of the plurality of historical voice samples of the subject.

In one or more embodiments, the plurality of voice samples may comprise an age and BMI matched dataset comprising voice samples from an equal number of T2DM sub-jects and non-diabetic subjects.

In one or more embodiments, each voice sample may comprise a predetermined phrase vocalized by the corresponding subject.

In one or more embodiments, the predetermined phrase may be displayed to the subject on a display device of a user device and each voice sample is recorded using an audio input device of the user device.

In one or more embodiments, the memory may further include: at least one selected from the group of: vocal parameter data for the plurality of subjects, age data of the plurality of subjects, and Body Mass Index (BMI) data of the plurality of subjects; and wherein the generating at the processor, the diabetic status model may be further based on the at least one selected from the group of: vocal parameter data for the plurality of subjects, age data of the plurality of subjects, and Body Mass Index (BMI) data of the plurality of subjects.

In one or more embodiments, the diabetic status prediction model may comprise at least one selected from the group of a Logistic Regression (LR) model, a Naïve Bayes (NB) model, and a Support Vector Machine (SVM) model.

In one or more embodiments, the diabetic status prediction model may comprise an ensemble model. This may include, averaging all the prediction probabilities within an individual, averaging the voice prediction results with the T2DM prevalence at the participant's age, averaging the voice prediction results with the T2DM prevalence at the participant's BMI, and/or a combination of these methods.

In one or more embodiments, the method may further include splitting the data. This can include the segmentation of data by participant ID into an age- and BMI-matched dataset (referred to as the “matched dataset”) for both males and females.

The present invention has been described here by way of example only. Various modification and variations may be made to these exemplary embodiments without departing from the spirit and scope of the invention, which is limited only by the appended claims.

All publications, patents and patent applications are herein incorporated by reference in their entirety to the same extent as if each individual publication, patent or patent application was specifically and individually indicated to be incorporated by reference in its entirety.

### EXAMPLES

### Example 1: Acoustic Analysis and Prediction of Type 2 Diabetes Mellitus Using Smartphone-Recorded Voice Segments

A study was performed to investigate whether occurrence of T2DM (including various T2DM states) could be identified in the voice of healthy individuals as well as methods for identifying voice biomarkers and associated models for generating predictive models. Participants were recruited from 4 sites in India, and were diagnosed by a physician as “Non-Diabetic” or “Type 2 Diabetic” according to guidelines set by the American Diabetes Association (ADA) (American Diabetes Association Professional Practice Committee, 2022). Individual participants recorded their own voices using a typical smartphone at several times throughout the day.

### Methods

### Participants and Study Design

Participants were recruited as part of a larger study involving the relationship between voice and glucose control. Participants were recruited from 4 sites in India, and were diagnosed by a physician as “Non-Diabetic” or “Type 2 Diabetic” according to guidelines set by the American Diabetes Association (ADA) (American Diabetes Association Professional Practice Committee, 2022). All participants signed informed consent. The study received full ethics clearance, and all methods were conducted in accordance with relevant guidelines and regulations (Type 2 diabetes glucose biomarker study with a continuous glucose monitoring system, 2020). Participants were instructed to record their voice at least six times a day for two weeks into a custom mobile application, saying the fixed sentences “Hello, how are you? What is my glucose level right now?”. Voice recordings were submitted and uploaded to a secure cloud database, where they were accessed by our researchers. All participants had no diagnosed neurological or speech disorder. A total of 267 participants were included (170 males—113 non-diabetic, 57 T2DM; and 97 females-79 non-diabetic and 18 T2DM), recording a total of 18465 voice samples.

### Data Split

To evaluate voice changes as a result of T2DM status (and not of confounding factors such as age or BMI), the data was segmented by participant ID into an age- and BMI-matched dataset (referred to as the “matched dataset”) for both males and females. Due to a large proportion of non-diabetic participants that skewed younger than the T2DM sample, a number of non-diabetic recordings that had no age and BMI match in the T2DM arm were excluded from the matched sample. An equal number of T2DM and non-diabetic participants were included in the matched dataset such that 50% of T2DM males and 61% of T2DM females were included. Statistical analysis and prediction model training were performed on the matched dataset, and the remaining data was used to test the fully trained model (referred to as the “test dataset”). The increased proportion of T2DM females was to allow for a sufficient number of recordings for analysis, while leaving sufficient data for testing the trained prediction model. In total, there were 4011 recordings and 1008 recordings for males and females, respectively, in the matched samples.

### Feature Extraction

Voice features were extracted using Parselmouth, a publicly available Python integration for Praat, a voice and speech analysis software (Jadoul et al., 2018, Boersma et al., 1996). A total of 14 voice features were extracted from each audio recording including pitch, pitch standard deviation, intensity, intensity standard deviation, harmonic noise ratio, 5 features corresponding with shimmer, and 4 features corresponding with jitter. Labels and description of voice features can be found in Appendix A.

Increased absolute values of shimmer and jitter are associated with increased perceived breathiness, hoarseness, and roughness in the voice, which can be linked to certain pathologies. These features are typically used exclusively in sustained phonation of vowel sounds, however they have been found to be useful in identifying dysphonia when calculated from an entire sentence recording (Ancillao et al., 2013). For this reason, jitter and shimmer values were chosen to be evaluated in addition to the pitch, intensity and harmonic noise ratio vocal parameters.

### Model

Although the voice dataset was large (18465 recordings), there were only 267 total participants. To account for the moderate number of participants, only models that traditionally perform well with small datasets were used to increase the likelihood of generalizability. As such, the probabilistic classification models Logistic Regression (LR), Gaussian Naïve Bayes (NB), and Support Vector Machine (SVM) were selected as models for the analysis. All model training, testing, and validation was performed in Python, using the Scikit-learn library (version 1.2.0, Python version 3.10.8).

### Feature Selection

Features were selected for inclusion in the models based on the Cohen's d effect size in the age matched data set. Cohen's d was calculated based on Equation 1, where the pooled standard deviation is the square root of the average variances of group A and B.

$$Cohen ' s d = \frac{Group A Mean - Group B Mean}{Pooled Standard Deviation} \qquad Equation ( 1 )$$

Features were iteratively added to the models based on decreasing absolute value of Cohen's d calculated from the matched dataset. Selected features were only added to the featureset if they were significantly different between the non-diabetic group and the diabetic group in both the matched sample and the entire dataset (i.e. P<0.05 after statistical analysis for the entire dataset and the matched dataset).

### Model Training and Cross-Validation

5-fold cross validation was performed on the matched dataset to find the improved model, featureset and threshold for prediction. All recordings from an individual were placed into the same fold.

After cross validation, the entire matched dataset was used for model training. All recordings corresponding to individuals within the matched dataset were used. The model performance was assessed on the testing set (i.e. the data not used in the matched dataset) using the pretrained model, feature set and threshold determined through cross validation of the matched data.

### Model Ensemble

After the prediction results for each recording were obtained, there were a few methods used to improve the accuracy of the predictions. All probabilities from all the prediction results for an individual were averaged to remove variability that may have occurred in the data collection process. Furthermore, the prediction probabilities were also averaged with the prevalence of T2DM at the participant's age and BMI (Daousi et al., 2006, Tandon et al., 2018). Finally, combinations of these averaging methods were used, and the models were compared to identify the improved ensemble method for both males and females.

### Statistical Analysis

Student's independent two-tailed t-test was performed on demographic and vocal parameter values between the group with T2DM and the non-diabetic group. Statistical analysis was performed in Python, using the SciPy package (version 1.9.3). Statistical significance is defined as P<0.05.

Model accuracy was assessed based on the accuracy, sensitivity and specificity of the trained models. To prevent overdiagnosis and false positives, only models with specificity >0.7 were considered to be the “improved” model.

### Results

### Participant Demographic Information

Demographic information of the entire dataset and the age- and bmi-matched dataset can be found in Table 1.

TABLE 1
Demographic information for study participants.

| Entire Dataset | | | Matched Dataset | | |
|---|---|---|---|---|---|
| ND | T2DM | p | ND | T2DM | p |

Female

| | | | | | | |
|---|---|---|---|---|---|---|
| Number of Participants | 79 | 18 | — | 11 | 11 | — |
| Total Number of Recordings | 5636 | 1183 | — | 503 | 505 | — |
| Age | 32.66 ± 10.85 | 28.20 ± 9.52 | <0.001 | 45.73 ± 10.47 | 45.91 ± 10.85 | 0.97 |
| BMI | 28.20 ± 9.52 | 35.09 ± 11.36 | 0.009 | 29.09 ± 5.29 | 31.41 ± 5.40 | 0.32 |
| Number of Recordings | 71.34 ± 23.75 | 65.72 ± 25.39 | 0.37 | 77.54 ± 10.56 | 70.91 ± 20.39 | 0.35 |
| per Participant | | | | | | |

Male

| | | | | | | |
|---|---|---|---|---|---|---|
| Number of Participants | 113 | 57 | — | 29 | 29 | — |
| Total Number of Recordings | 7935 | 3711 | — | 1964 | 2047 | — |
| Age | 32.73 ± 11.36 | 48.96 ± 10.03 | <0.001 | 45.00 ± 10.92 | 44.76 ± 10.61 | 0.38 |
| BMI | 27.69 ± 7.54 | 30.87 ± 14.33 | <0.001 | 27.77 ± 4.46 | 26.7 ± 3.28 | 0.19 |
| Number of Recordings | 70.22 ± 23.23 | 65.11 ± 36.78 | 0.27 | 67.72 ± 33.27 | 70.59 ± 25.40 | 0.72 |
| per Participant | | | | | | |

Values are displayed as mean ± standard deviation.
P-values are independent two-tailed student's t test.
Bolded values indicate statistical significance (P < 0.05).

### Feature Extraction and Statistical Analysis

14 voice features were extracted from each voice recording. Pitch, pitch standard deviation, HNR, apq3Shimmer, apq11Shimmer, ddaShimmer, localabsoluteJitter, rapJitter, and ddpJitter were significant between the non-diabetic and diabetic females in both the matched dataset and the entire dataset. Pitch standard deviation, mean intensity, intensity standard deviation, local Shimmer, local absolute Shimmer, apq5 shimmer, apq11 shimmer, local jitter, localabsoluteJitter, rapJitter, ppq5 Jitter, and ddpJitter were significant between the non-diabetic and diabetic males in both the matched dataset and the entire dataset (Table 2).

### Model Training and Threshold Determination

Only the matched dataset was used in model training and the threshold determination to avoid any confounding factors from the demographics in the model training. 5-fold cross validation was used to evaluate model performance and identify the improved threshold for the data. Data was split into the training and testing data based off of an individual's ID and not by the recording themselves. After training, the model was applied to each recording in the testing set to individually predict diabetic status. Three models were implemented—Gaussian Naïve Bayes, Logistic Regression, and Support Vector Machine, and performance of the models were evaluated using one, two, three, and four features in the featureset. These features were selected from the features that were statistically significant in both datasets, using the features with the largest Cohen's d in the matched dataset (Table 2). The features used in the model evaluation were, in order of addition, pitch standard deviation, mean pitch, rap jitter, and apq3 shimmer for females and mean intensity, apq11 shimmer, intensity standard deviation, and ppq5 jitter for males.

TABLE 2
Acoustic Analysis of All Audio Recordings.

| | Entire Dataset | | | | Matched Dataset | | | |
|---|---|---|---|---|---|---|---|---|
| Feature | ND | T2DM | p | Cohen's d | ND | T2DM | p | Cohen's d |

Female

| | | | | | | | | |
|---|---|---|---|---|---|---|---|---|
| meanF0 | 213.23 ± 34.49 | 191.73 ± 37.38 | <0.001 | 0.6 | 210.03 ± 37.22 | 194.65 ± 37.13 | <0.001 | 0.41 |
| stdevF0 | 38.66 ± 19.97 | 32.88 ± 19.63 | <0.001 | 0.29 | 44.58 ± 21.19 | 34.11 ± 19.99 | <0.001 | 0.51 |
| meanInten | 65.39 ± 6.33 | 65.6 ± 5.61 | 0.3 | 0.04 | 64.72 ± 5.65 | 66.46 ± 4.65 | <0.001 | 0.34 |
| stdevInten | 7.7 ± 2.96 | 8.76 ± 3.43 | <0.001 | 0.33 | 8.43 ± 3.03 | 8.59 ± 3.26 | 0.29 | 0.05 |
| HNR | 12.1 ± 3.95 | 11.53 ± 4.32 | <0.001 | 0.14 | 11.15 ± 3.79 | 11.74 ± 4.42 | 0.004 | 0.14 |
| localShimmer | 0.12 ± 0.04 | 0.12 ± 0.03 | 0.37 | 0.03 | 0.12 ± 0.04 | 0.12 ± 0.04 | <0.001 | 0.2 |
| localdbShimmer | 1.13 ± 0.27 | 1.13 ± 0.25 | 0.5 | 0.02 | 1.19 ± 0.27 | 1.13 ± 0.27 | <0.001 | 0.24 |
| apq3Shimmer | 0.05 ± 0.02 | 0.05 ± 0.02 | 0.002 | 0.1 | 0.06 ± 0.02 | 0.05 ± 0.02 | <0.001 | 0.23 |
| apq5Shimmer | 0.07 ± 0.03 | 0.07 ± 0.03 | 0.09 | 0.06 | 0.08 ± 0.03 | 0.07 ± 0.03 | <0.001 | 0.24 |
| apq11Shimmer | 0.11 ± 0.04 | 0.11 ± 0.03 | 0.02 | 0.08 | 0.12 ± 0.04 | 0.11 ± 0.04 | <0.001 | 0.2 |
| localJitter | 0.02 ± 0.01 | 0.02 ± 0.01 | 0.86 | 0.01 | 0.02 ± 0.01 | 0.02 ± 0.01 | <0.001 | 0.44 |
| localabsoluteJitter | 0.00010 ± 0.00005 | 0.00012 ± 0.00005 | <0.001 | 0.24 | 0.00012 ± 0.00005 | 0.00011 ± 0.00005 | 0.03 | 0.11 |
| rapJitter | 0.011 ± 0.004 | 0.010 ± 0.004 | 0.02 | 0.08 | 0.012 ± 0.004 | 0.010 ± 0.004 | <0.001 | 0.4 |
| ppq5Jitter | 0.011 ± 0.004 | 0.011 ± 0.004 | 0.97 | 0.001 | 0.013 ± 0.004 | 0.011 ± 0.004 | <0.001 | 0.41 |

Male

| | | | | | | | | |
|---|---|---|---|---|---|---|---|---|
| meanF0 | 140.04 ± 29.71 | 140.2 ± 32.79 | 0.8 | 0.01 | 141.4 ± 33.7 | 139.35 ± 33.91 | 0.06 | 0.06 |
| stdevF0 | 25.34 ± 23.48 | 28.78 ± 25.56 | <0.001 | 0.14 | 29.29 ± 25.6 | 29.49 ± 26.73 | 0.81 | 0.01 |
| meanInten | 67.14 ± 6.70 | 62.64 ± 8.08 | <0.001 | 0.61 | 65.87 ± 6.29 | 62.88 ± 7.90 | <0.001 | 0.42 |
| stdevInten | 8.00 ± 3.56 | 8.92 ± 3.00 | <0.001 | 0.28 | 8.34 ± 2.89 | 9.02 ± 2.96 | <0.001 | 0.23 |
| HNR | 10.61 ± 3.34 | 10.76 ± 3.12 | 0.02 | 0.05 | 10.53 ± 3.11 | 10.53 ± 3.01 | 0.95 | 0.002 |
| localShimmer | 0.12 ± 0.03 | 0.13 ± 0.03 | <0.001 | 0.19 | 0.12 ± 0.03 | 0.13 ± 0.03 | <0.001 | 0.15 |
| localdbShimmer | 1.16 ± 0.23 | 1.21 ± 0.22 | <0.001 | 0.25 | 1.18 ± 0.22 | 1.22 ± 0.21 | <0.001 | 0.17 |
| apq3Shimmer | 0.05 ± 0.02 | 0.05 ± 0.02 | 0.02 | 0.05 | 0.05 ± 0.02 | 0.05 ± 0.02 | 0.52 | 0.02 |
| apq5Shimmer | 0.07 ± 0.02 | 0.08 ± 0.02 | <0.001 | 0.2 | 0.08 ± 0.02 | 0.08 ± 0.02 | 0.003 | 0.09 |
| apq11Shimmer | 0.12 ± 0.04 | 0.14 ± 0.04 | <0.001 | 0.35 | 0.13 ± 0.04 | 0.14 ± 0.04 | <0.001 | 0.28 |
| localJitter | 0.02 ± 0.01 | 0.02 ± 0.01 | <0.001 | 0.24 | 0.022 ± 0.007 | 0.024 ± 0.007 | <0.001 | 0.2 |
| localabsoluteJitter | 0.00016 ± 0.00006 | 0.00018 ± 0.00006 | <0.001 | 0.21 | 0.00017 ± 0.00007 | 0.00018 ± 0.00007 | <0.001 | 0.19 |
| rapJitter | 0.011 ± 0.004 | 0.011 ± 0.004 | <0.001 | 0.17 | 0.011 ± 0.004 | 0.011 ± 0.004 | <0.001 | 0.15 |
| ppq5Jitter | 0.012 ± 0.004 | 0.013 ± 0.004 | <0.001 | 0.22 | 0.012 ± 0.004 | 0.013 ± 0.004 | <0.001 | 0.22 |

P-values from independent two-tailed student's t test.
Bolded values indicate features used in model development (i.e. statistical significance (P < .05) between non-diabetic and T2DM for both the entire dataset and the matched dataset and Cohen's d effect size >0.2 in the matched dataset).

### Female Model Training Results

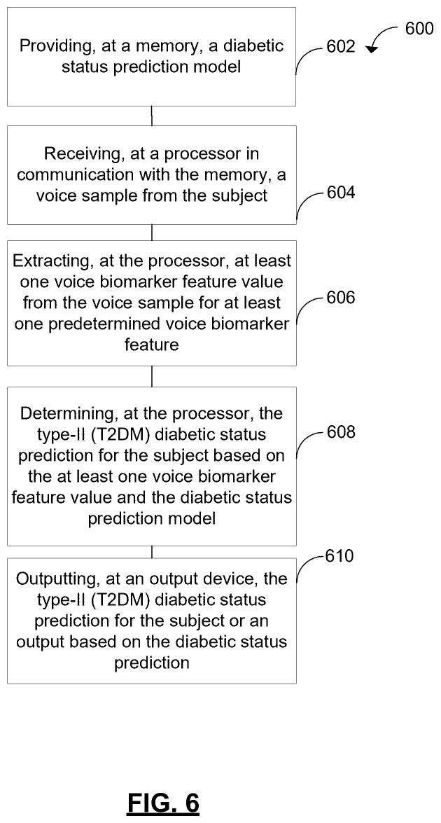

The improved model for females after cross validation was a 3-feature LR model and had a specificity of 0.75±0.15, a sensitivity of 0.54±0.14, and an improved threshold of 0.54 for all data recordings (Table 3, FIG. 8). The three features used in the female model were the mean pitch, pitch standard deviation, and the relative average perturbation of the jitter (rap jitter). If all recordings for a participant were averaged, the 3-feature LR female model had a specificity of 0.90±0.22 and a sensitivity of 0.54±0.07 for the threshold of 0.54 (Table 3).

### Male Model Training Results

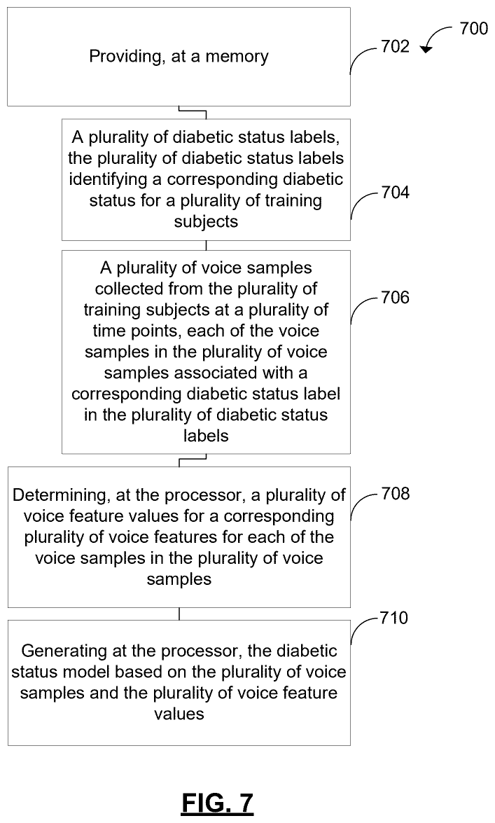

The improved model for males was a 2-feature NB and had a specificity of 0.70±0.13, a sensitivity of 0.49±0.09, and an improved threshold of 0.46 for all data recordings (Table 3, FIG. 9). The features used in the male model were the mean intensity and apq11 shimmer values. If the results were averaged for each participant, the 2-feature NB male model had a specificity of 0.70±0.19 and a sensitivity of 0.59±0.16 with a 0.46 threshold (Table 3).

### Model Prediction Results

The model was retrained using the entire matched dataset as the training data. Model validation was performed on the data that was not included in the matched dataset, using the same improved model and threshold as determined by cross validation of the training data.

### Female Model Prediction Results

The female validation set had a final specificity of 0.71 and sensitivity of 0.58 (3-feature LR model), and if prediction results for all recordings for a participant were averaged, the 3-feature LR model had a specificity of 0.87 and sensitivity of 0.57 (Table 3).

### Male Model Prediction Results

The male validation set had a final specificity of 0.74 and sensitivity of 0.52 when predicting individual recordings (2-feature NB model), and if all the prediction results for an individual were averaged, the 2-feature NB model had a specificity of 0.75 and sensitivity of 0.54 (Table 3).

### Incorporation of Demographic Data

To increase model accuracy, age and BMI were incorporated into the prediction methodology. Age was converted to the prevalence of T2DM at the given age (Tandon et al., 2018), and BMI was converted to T2DM prevalence at the participant's BMI (Daousi et al., 2006).

These prevalence values were averaged with the percent likelihood model prediction of T2DM in three different ensemble prediction methods:
1. Age T2DM prevalence averaged with voice prediction results,
2. BMI T2DM prevalence averaged with voice prediction results, and
3. Age and BMI T2DM prevalence averaged with voice prediction results.

New thresholds for prediction were determined through 5-fold cross validation of the matched dataset, but the features used in prediction and the model type were kept the same as the improved model determined in the voice-only prediction. The models were validated on the remaining (non-matched) data, using the matched dataset for model training and the threshold determined by cross validation.

### Female Ensemble Results

The improved ensemble model prediction was achieved by averaging the female voice recording results with the BMI prevalence of T2DM. The model had an improved accuracy of 0.75±0.22, with a specificity of 0.77±0.29 and a sensitivity of 0.73±0.23 from cross validation of the matched dataset, and had an accuracy of 0.89, specificity of 0.91, and sensitivity of 0.71 when predicting the test set (Table 3).

### Male Ensemble Results

The improved ensemble model prediction was obtained by averaging the male voice recording prediction results with the age and BMI prevalence of T2DM. This model had an improved accuracy of 0.70±0.10, with a specificity of 0.73±0.13 and a sensitivity of 0.69±0.15 from cross validation of the matched dataset, and had an accuracy of 0.86, specificity of 0.89, and sensitivity of 0.75 when predicting the test set (Table 3)

TABLE 3
Model performance results for the cross validation and testing results. 5-fold cross validation was performed on the matched dataset,
and the model testing was performed on the testing dataset.

| Model | | | | s-fold CV | | | | Testing | | | |
|---|---|---|---|---|---|---|---|---|---|---|---|
| Ensemble | Type | Model | Threshold | Sensitivity | Specificity | Accuracy | BCA | Sensitivity | Specificity | Accuracy | BCA |

Female

| | | | | | | | | | | | |
|---|---|---|---|---|---|---|---|---|---|---|---|
| Just Voice | All Recordings | 3-feature LR | 0.54 | 0.54 ± 0.14 | 0.75 ± 0.15 | 0.67 ± 0.14 | 0.65 ± 0.14 | 0.58 | 0.71 | 0.7 | 0.64 |
| Just Voice | Averaged per | 3-feature LR | 0.54 | 0.53 ± 0.07 | 0.90 ± 0.22 | 0.72 ± 0.13 | 0.72 ± 0.13 | 0.57 | 0.87 | 0.84 | 0.72 |
| | Individual | | | | | | | | | | |
| Voice and | All Recordings | 3-feature LR | 0.31 | 0.67 ± 0.18 | 0.70 ± 0.11 | 0.68 ± 0.13 | 0.67 ± 0.13 | 0.69 | 0.80 | 0.80 | 0.75 |
| Age | | | | | | | | | | | |
| Voice and | Averaged per | 3-feature LR | 0.3 | 0.63 ± 0.37 | 0.83 ± 0.21 | 0.73 ± 0.28 | 0.73 ± 0.28 | 0.71 | 0.75 | 0.75 | 0.73 |
| Age | Individual | | | | | | | | | | |
| Voice and | All Recordings | 3-feature LR | 0.31 | 0.69 ± 0.18 | 0.71 ± 0.24 | 0.70 ± 0.19 | 0.70 ± 0.19 | 0.73 | 0.71 | 0.71 | 0.72 |
| BMI | | | | | | | | | | | |
| Voice and | Averaged per | 3-feature LR | 0.3 | 0.73 ± 0.23 | 0.77 ± 0.29 | 0.75 ± 0.22 | 0.75 ± 0.22 | 0.71 | 0.91 | 0.89 | 0.81 |
| BMI | Individual | | | | | | | | | | |
| Voice, Age, | All Recordings | 3-feature LR | 0.24 | 0.70 ± 0.22 | 0.72 ± 0.19 | 0.71 ± 0.19 | 0.71 ± 0.19 | 0.79 | 0.83 | 0.82 | 0.81 |
| and BMI | | | | | | | | | | | |
| Voice, Age, | Averaged per | 3-feature LR | 0.24 | 0.63 ± 0.37 | 0.83 ± 0.21 | 0.73 ± 0.28 | 0.73 ± 0.28 | 0.71 | 0.93 | 0.91 | 0.82 |
| and BMI | Individual | | | | | | | | | | |

Male

| | | | | | | | | | | | |
|---|---|---|---|---|---|---|---|---|---|---|---|
| Just Voice | All Recordings | 2-feature NB | 0.46 | 0.49 ± 0.09 | 0.70 ± 0.13 | 0.60 ± 0.08 | 0.60 ± 0.09 | 0.52 | 0.74 | 0.69 | 0.63 |
| Just Voice | Averaged per | 2-feature NB | 0.46 | 0.59 ± 0.16 | 0.70 ± 0.19 | 0.66 ± 0.11 | 0.65 ± 0.11 | 0.54 | 0.75 | 0.70 | 0.64 |
| | Individual | | | | | | | | | | |
| Voice and | All Recordings | 2-feature NB | 0.29 | 0.56 ± 0.06 | 0.730.10 | 0.64 ± 0.03 | 0.65 ± 0.04 | 0.60 | 0.87 | 0.81 | 0.74 |
| Age | | | | | | | | | | | |
| Voice and | Averaged per | 2-feature NB | 0.28 | 0.58 ± 0.19 | 0.73 ± 0.24 | 0.66 ± 0.13 | 0.65 ± 0.13 | 0.82 | 0.87 | 0.86 | 0.85 |
| Age | Individual | | | | | | | | | | |
| Voice and | All Recordings | 2-feature NB | 0.28 | 0.58 ± 0.08 | 0.75 ± 0.11 | 0.66 ± 0.03 | 0.66 ± 0.03 | 0.56 | 0.75 | 0.71 | 0.65 |
| BMI | | | | | | | | | | | |
| Voice and | Averaged per | 2-feature NB | 0.28 | 0.59 ± 0.12 | 0.79 ± 0.16 | 0.69 ± 0.11 | 0.69 ± 0.11 | 0.57 | 0.76 | 0.72 | 0.67 |
| BMI | Individual | | | | | | | | | | |
| Voice, Age, | All Recordings | 2-feature NB | 0.22 | 0.58 ± 0.06 | 0.74 ± 0.11 | 0.67 ± 0.03 | 0.66 ± 0.03 | 0.66 | 0.84 | 0.8 | 0.75 |
| and BMI | | | | | | | | | | | |
| Voice, Age, | Averaged per | 2-feature NB | 0.22 | 0.69 ± 0.15 | 0.73 ± 0.13 | 0.70 ± 10.10 | 0.71 ± 0.11 | 0.75 | 0.89 | 0.86 | 0.82 |
| and BMI | Individual | | | | | | | | | | |

LR = Logistic Regression,
NB = Gaussian Naïve Bayes,
BCA = Balanced Class Accuracy,
BMI = Body Mass Index

### Discussion

Overall, there are distinct differences between the voices of individuals with and without T2DM. These differences vary between males and females, and reinforce prior findings that the vocal manifestations of diabetes are sex specific. The most accurate prediction method involved an ensemble model with T2DM prevalence at the participant's age and BMI for males and the participant's BMI for females, resulting in a maximum test accuracy of 0.89 for females and 0.86 for males.

In females, the features used in the prediction model with the largest Cohen's d value were the mean pitch, pitch standard deviation and the rap jitter. Mean pitch and pitch standard deviation decreased in females with T2DM compared to females without T2DM. This effect has been seen before, in which women's health risk factors such as weight and BMI were negatively correlated with pitch (Ravishankar et al., 2020, Souza et al., 2018). As these health risk factors are highly associated with T2DM (American Diabetes Association Professional Practice Committee, 2022), it stands to reason that pitch would also decrease for females with T2DM.

In males, the features with the highest predictive ability and largest Cohen's d value were the mean intensity and the apq11 shimmer. Mean intensity decreases with T2DM compared to non-diabetic individuals, indicating that T2DM individuals may have a weaker or less powerful voice compared to their non-diabetic counterparts. Apq11 shimmer increased with T2DM compared to non-diabetic individuals, indicating more variation in the amplitudes of a voice sample and increased vocal instability. In previous studies, T2DM was associated with higher self reported hoarseness and vocal strain, and diabetic peripheral neuropathy was associated with self reported loss of voice or aphonia (Hamdan et al., 2012). Muscle weakness and atrophy can occur in individuals with T2DM (Perry et al., 2016), which may be reflected as weakness or instability in the vocal cords.

The model accuracy increases when the voice samples from a single individual are averaged, most likely a result of eliminating any variation that may occur between recordings within an individual. This effect is primarily seen from an increase in the specificity. Sensitivity of the prediction model, on the other hand, does not tend to increase when averaging recordings of T2DM individuals. This may have to do with the fact that only approximately 50% of T2DM cases experience peripheral neuropathy or other symptoms associated with neurological and vascular damage (Hicks et al., 2019). This aligns with the current prediction methodology, as 57% of females and 46% of males in the testing set were correctly predicted to be T2DM. Some individuals would not have any complications related to vocal changes from T2DM, and therefore would not be detected by averaging the voice recording prediction results.

An interesting outcome of the analysis is that prediction of T2DM exclusively from voice is slightly more sensitive in females than males in the testing set (57% vs 46%). This result is unlikely to be due to peripheral neuropathy, as peripheral neuropathy prevalence, onset, and severity is increased in males compared to females (Hicks et al., 2019). An alternate explanation for the discrepancy is the decrease in cognitive function in females with T2DM compared to males, in both individuals with diabetic peripheral neuropathy and without diabetic peripheral neuropathy (Palomo-Osuna et al., 2022). Cognitive impairment has been shown to have a significant effect on the voice, with strong predictive capabilities (López-de-Ipiña et al., 2020), providing evidence in support of distinguishing between females with and without T2DM. Furthermore, females are at a higher risk of Major Depressive Disorder (MDD) than males when diagnosed with T2DM (Deischinger et al., 2020), a disorder that has also been linked to voice changes such as slower speech and a lower pitch (Wang et al., 2019). Future work should incorporate metrics of cognitive impairment, mental health reports, and the degree of peripheral neuropathy to the data collection to confirm these findings. Sex differences in T2DM have become increasingly prominent, so future research should carefully account for these differences, along with gender disparities, for a more comprehensive insight.

Age and BMI have been associated with vocal changes in previous work (Ravishankar et al., 2020, Souza et al., 2018), so analysis of a sub-dataset was performed to ensure that results were associated with diabetes presence and not other external factors. Incorporating vocal parameters, age, and BMI into an ensemble prediction model was able to achieve over 70% accuracy in an age- and bmi-matched dataset, and achieved even higher accuracy in the unmatched dataset. This result indicates that even the simple incorporation of age and BMI into an ensemble model with voice creates an accurate prediction methodology for T2DM.

Although voice changes in T2DM have been previously studied, previous work has primarily used a stand-alone microphone or specific recording device to collect data rather than using an smartphone and app-based approach. App-based recording offers significant advantages over stand-alone microphones for voice data collection. Its accessibility options allow researchers to capture data using widely available devices, expanding participant inclusivity. Additionally, smartphone recording has the potential to capture real-world situations, capturing speech and interactions in familiar surroundings. Overall, researchers can obtain insights into the voice changes in everyday scenarios.

There were some limitations to the presented methodology. The duration of T2DM may have an effect on the voice (Hamdan et al., 2012), and future work should incorporate the collection of T2DM duration into the study protocol. Additionally, the feature set used in the analysis was small (N=14 features) to allow for discussion and interpretation of the results. There may be more accurate prediction results when there is an expanded feature set such as what is seen in OpenSmile (Eyben et al., 2010), particularly looking at intensity and shimmer values for males and pitch and jitter values for females. With more features, more complex models may be implemented and evaluated such as random forest models or neural networks. Finally, only a crude ensemble of demographic and vocal features was used in the final model implementation. Future work could explore alternative ways to incorporate demographic data into the model results.

The material presented here indicates there is strong evidence for utilizing voice analysis for the detection of T2DM. With the success of this approach, the results may even be expanded for the use of voice for prediabetes detection to provide an easy and inexpensive screening method or monitoring tool. With the increasing prevalence of T2DM, leveraging the potential of voice analysis as an accessible and cost-effective screening tool becomes imperative. This approach holds promise not only for T2DM detection but also for addressing the rising burden of the disease, providing an efficient means of early intervention and management.

### APPENDIX A—ACOUSTIC VOICE FEATURES

TABLE A1
Acoustic voice features extracted from audio samples.

| Feature | Description |
|---|---|
| meanF0 | Mean pitch value for the voice recording. |
| stdevF0 | Standard deviation of the pitch for the voice recording. |
| meanInten | Mean intensity for the voice recording. |
| stdevInten | Standard deviation of the intensity for the voice recording. |
| HNR | Harmonic-to-Noise Ratio, or Harmonicity. Represents the degree of acoustic periodicity in |
| | a signal and measures the ratio between the energy in the harmonics and the noise, |
| | expressed in dB. Higher HNR values indicate more energy in the harmonics. |
| localShimmer | The average difference between the amplitudes of consecutive periods, divided by |
| | the average amplitude. |
| localdbShimmer | The average logarithm (base-10) of the difference between the amplitudes of consecutive |
| | periods, multiplied by 20. |
| apq3Shimmer | Amplitude Perturbation Quotient for three points. It measures the average difference |
| | between the amplitude of a period and the average amplitude of its neighboring periods, |
| | divided by the average amplitude. |
| apq5Shimmer | Amplitude Perturbation Quotient for five points. It determines the average difference |
| | between the amplitude of a period and the average amplitude of the period itself and |
| | its four closest neighbors, divided by the average amplitude. |
| apq11Shimmer | Amplitude Perturbation Quotient for eleven points. It computes the average difference |
| | between the amplitude of a period and the average amplitude of the period itself and |
| | its ten closest neighbors, divided by the average amplitude. |
| localJitter | The average difference between consecutive periods, divided by the average period. |
| localabsJitter | The average absolute difference between consecutive periods in seconds. |
| rapJitter | Relative Average Perturbation, which is the average absolute difference between a period |
| | and the average of the period itself and its two neighboring periods, divided by the |
| | average period. |
| ppq5Jitter | Five-point Period Perturbation Quotient, which represents the average absolute |
| | difference between a period and the average of the period itself and its four closest |
| | neighbors, divided by the average period. |

### Example 2: Improving Voice Sample Quantity and Recording Settings for Enhanced T2DM Prediction Accuracy

### Introduction

Diabetes mellitus (DM) is a chronic metabolic disorder characterized by persistent elevated blood glucose levels due to inadequate insulin production or impaired insulin utilization. It affects 10.5% of the population (536.6 million people) (H. Sun) with type 2 (T2DM) accounting for 90% of all cases (R. Goyal). Uncontrolled DM ranks among the primary contributors to cardiovascular disorders, blindness, renal failure, and lower limb amputation (R. Goyal). Early detection and effective management of diabetes play a pivotal role in mitigating the onset and progression of complications associated with the condition, thereby significantly reducing the risk of long-term health complications and enhancing overall quality of life.

Traditional methods for diagnosing diabetes, that is fasting plasma glucose and oral glucose tolerance tests, involve blood sampling which can pose inconvenience or discomfort to patients, rendering it inadequate for frequent monitoring of the condition. Moreover, lack of glucometer and the time spent for self-testing are barriers in self-management of diabetes (V. Mogre, J. S. Gonzalez). In recent years, there has been a growing interest in the use of voice biomarkers as promising non-invasive and convenient alternatives for diagnosing and monitoring diabetes, particularly beneficial for individuals residing in remote communities with restricted healthcare accessibility.

Acoustic biomarkers extracted from speech signals reflect underlying physiological and pathological states. The sustained periods of high blood glucose and detrimental effects of peripheral neuropathy and myopathy in individuals with T2DM, impact the elastic properties of the vocal cords (Sidorova) and weaken muscle within the larynx, resulting in voice disorders like hoarseness (Hamdan 2013) and dysphagia (Weinreb). As a result, there are significant vocal differences between individuals with T2DM and those without the condition, providing strong justification for investigating vocal variations in T2DM (Pinyopodjanard, Gölaç, Chitkara).

In line with this pursuit, embodiments herein previously assessed the feasibility of voice recording using mobile applications for prediction and detection of T2DM (Kaufman). Embodiments herein and associated Example results showed that voice biomarkers along with age and BMI are able to improve T2DM prediction with accuracy Of 0.89 for women and 0.86 for men (Kaufman). Building upon that study, where participants were tasked with recording their voices at least 6 times daily over a 2-week period, it became apparent that such a regimen could lead to participant burden and inconsistency in recordings. Therefore, in this study, our goal was to streamline this process by determining the 1) improved number of voice samples, and 2) improved recording schedule (frequency & timing), which is required to maintain T2DM diagnostic efficacy while reducing patient burden.

### Methods

To achieve the objectives of this study, the data from 78 participants (18+ years old, 22 women) was retrospectively analyzed. The analysis pipeline included 1) investigating the variances in the predictive model across varying quantities of voice samples, 2) analysis of diverse recording configurations to ascertain their impact on predictive performance metrics. The study was approved by ethics and followed the guidelines and regulations.

### Participants

Participants were selected from a larger cohort, recruited from 4 sites in India, (Kaufman) to ensure matching of age and body mass index (BMI), thereby minimizing the demographic impact on voice recordings (Table 4). Participants were diagnosed by a physician as either non-diabetic or T2DM following the American Diabetes Association (ADA) guidelines (American Diabetes Association Professional Practice Committee). Participants were nonsmokers and had no diagnosed neurological or speech impairments. All participants signed consent form.

### Measurements

Following the study protocol, over a two-week period, participants recorded their voice at least 6 times per day using a custom mobile application while repeating the phrases: “Hello, how are you? What is my glucose level right now?”. The start day (d01) for each participant was considered the day in which at least 2 voice samples were recorded.

### Data Processing

Using the model developed by our team (Kaufman), the probability of T2DM was calculated for each recording. Given multiple voice recordings, a participant was classified as having T2DM if the average estimated probability of the model for a single voice sample equaled or exceeded a set threshold (0.215 for men and 0.3 for women). The accuracy of the model was estimated by the number of correct classification of participants.

To indicate the improved number of voice recordings to achieve effective T2DM diagnostic, the accuracy of the model was calculated for different number of voice recordings (n). To do so, participants were first identified who have at least n voice samples among all days. Then, n samples were randomly selected for each participant and identified T2DM class based on the average estimated probability. The accuracy of the model was estimated based on the number of correct predictions. This process was repeated 2000 times to encompass a wide range of combinations of voice samples per participant.

To examine the variability of diagnostic accuracy across different days using n voice recordings, participants were identified who have at least n voice samples in each day. Then, T2DM was predicted for each combination of n voice recordings for each participant. The accuracy of the model was estimated based on the number of correct predictions for each participant. The overall accuracy was estimated as the average accuracy of the model over all participants.

Finally, to investigate the effect of deviation from recording of all voice samples in one day, the model accuracy was calculated using different distribution of voice recordings across 2 first days. To do so, participants were identified who have at least n1 voice samples in day 1 and n−n1 voice samples in day 2. Then, for each participant, the average model probability was calculated given all combinations of voice recordings across 2 days. The accuracy of the model was estimated based on the number of correct predictions for each participant. The overall accuracy was estimated as the average accuracy of the model over all participants.

To study the changes in the trend of the model accuracy using a breakpoint of n samples, segmented linear regression was employed by fitting two distinct linear models to the data before and after the breakpoint. Kendall's Tau measure of correlation was used to investigate the strength and direction of the relationship between ordinal variables (such as days) and model accuracy. P-value of 0.05 were considered statistically significant.

### Results

Participants demographics are presented in Table 4. Participants of the 2 group (non-diabetic, and T2DM) were matched based on age and BMI.

### Participant Demographics

TABLE 4
Patients' Demographics

| | Total | Non-Diabetic | T2DM |
|---|---|---|---|
| N (%) | 78 (100.0) | 39 (50.0) | 39 (50.0) |
| Women | 22 (28.21) | 11 (50.0) | 11 (50.0) |
| Men | 56 (71.79) | 28 (50.0) | 28 (50.0) |
| Age (years) | 45.26 ± 10.63 | 45.49 ± 10.8 | 45.03 ± 10.58 |
| Women | 45.82 ± 10.4 | 45.91 ± 10.85 | 45.73 ± 10.47 |
| Men | 45.04 ± 10.8 | 45.32 ± 10.98 | 44.75 ± 10.8 |
| BMI (kg/m²) | 28.07 ± 4.59 | 28.77 ± 5.01 | 27.36 ± 4.06 |
| Women | 30.25 ± 5.35 | 31.41 ± 5.4 | 29.09 ± 5.29 |
| Men | 27.21 ± 3.98 | 27.74 ± 4.53 | 26.68 ± 3.34 |

N: Number,
BMI: Body Mass Index
Continuous variables are presented with mean ± SD.
Categorical variables are summarized as frequency (percentage).

### Voice Recordings

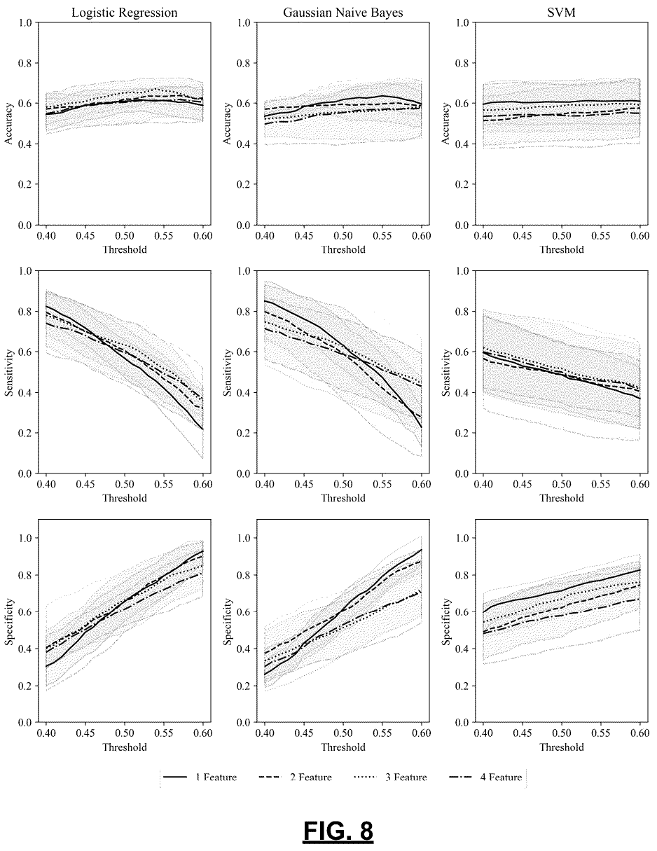

In total 5035 recordings were included in the analysis (Table 5). FIG. 10 presents the distribution of voice recordings per day for participants. The daily average recording for all participants was 4.91±1.45 with women more adherent to the protocol than men (average 5.13±1.38 vs. 4.82±1.46 recordings per day).

TABLE 5
Voice Recordings

| | Total | Non-Diabetic | T2DM |
|---|---|---|---|
| N (%) | 5035 | 2415 | 2620 |
| Women | 1539 | 713 | 826 |
| Men | 3496 | 1702 | 1794 |
| Number of recordings per participant | 64.55 ± 19.53 | 61.92 ± 21.67 | 67.18 ± 17.0 |
| Women | 69.95 ± 16.26 | 64.82 ± 19.12 | 75.09 ± 11.45 |
| Men | 62.43 ± 20.42 | 60.79 ± 22.82 | 64.07 ± 17.97 |

N: Number,
BMI: Body Mass Index
Continuous variables are presented with mean ± SD.
Categorical variables are summarized as frequency (percentage).

### Improved Number of Voice Recordings for Maximum Model Accuracy

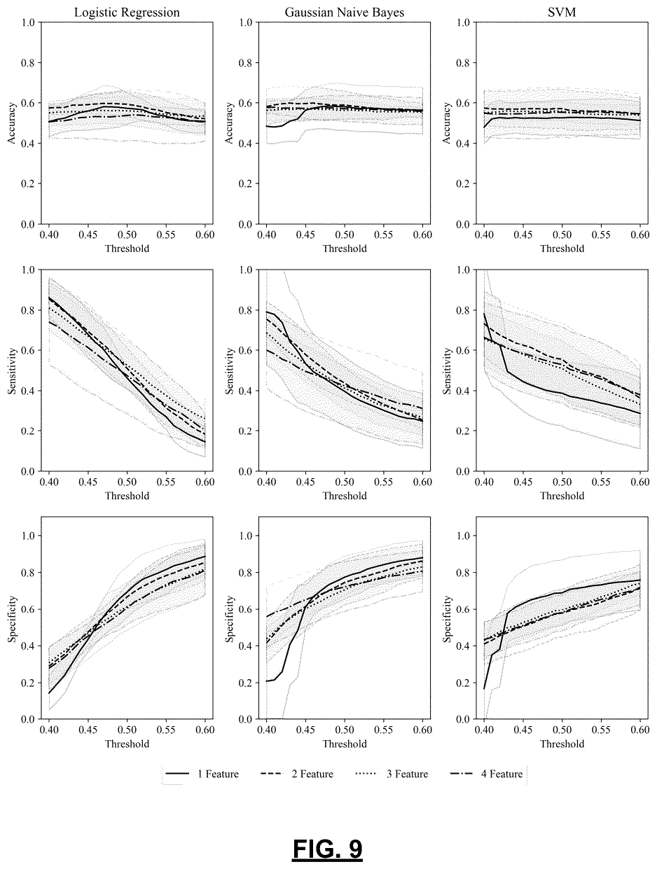

FIG. 11 presents the accuracy of the model using different number of voice recordings for men and women. Our results show that both in men and women, the accuracy of the model increases with the inclusion of up to six voice samples, after which it stabilizes with no significant improvement. The changes in slope of the linear fit are −1.15 for men and −0.65 for women, indicating that the model's accuracy improves more rapidly with the addition of initial voice samples in men compared to women. This suggests that while additional samples beyond six contribute minimally to accuracy for both genders, the rate of improvement is initially greater in men.

### Improved Settings of Voice Recordings for Maximum Model Accuracy

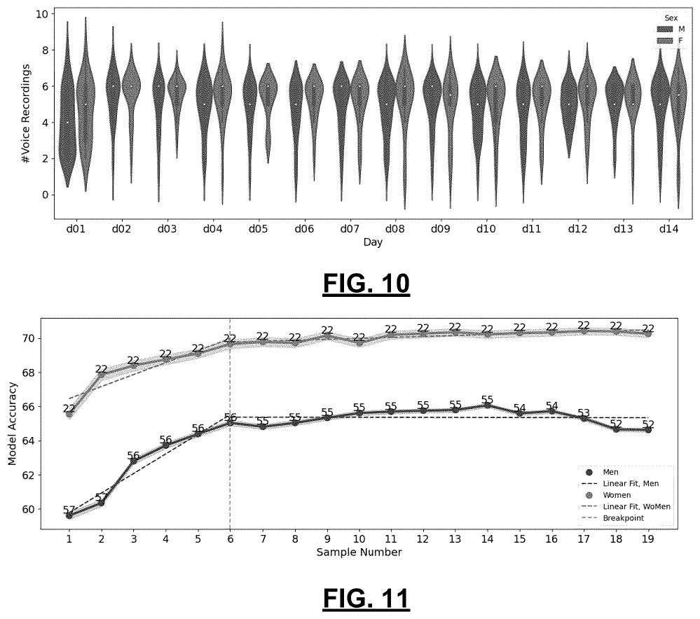

FIG. 12 presents the accuracy of the model using 6 samples recorded in different days. For men, the highest model accuracy was achieved with recordings from day 1, while for women, the peak accuracy was observed with recordings from day 10. However, the variations in model accuracy across different days were not significant, and no statistically discernible trend was observed (men: p=0.233; women: p=0.271). The model accuracy was generally higher for women than men on most days. However, the difference was not statistically significant, as evidenced by the overlap between the confidence intervals of accuracy for men and women. On days 7 and 10, the model accuracy was notably higher for women than men.

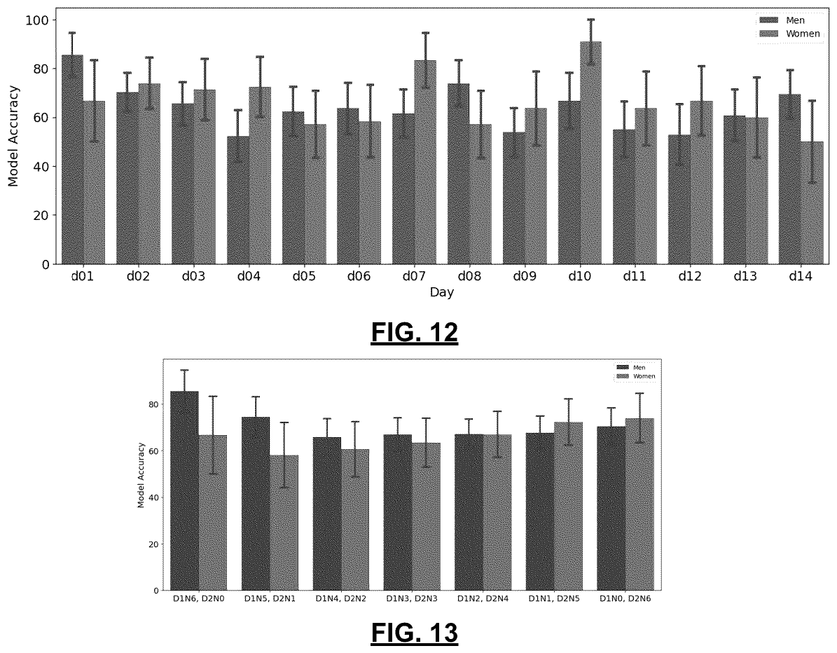

Given the variability in voice recordings across days between individuals (FIG. 10), the model accuracy was also examined using 6 voice recordings during first two days to explore potential deviations from the protocol of recording 6 samples in a single day (FIG. 13). Our results showed that the accuracy of model was higher in men than women when the majority of recordings were from first day. As the distribution of recordings balanced between the first and second days, the accuracy gap between men and women narrowed. Finally, when the majority of recordings were from the second day, the model's accuracy was slightly higher for women than men, with the differences in accuracy levels becoming less pronounced. Our statistical test indicated no significant trend in model accuracy for men when using 6 recordings across two days (p=1). The high value of accuracy observed for men on the first day could be attributed to the lower number of individuals with at least six recordings on that day. This smaller sample size may have led to less variability and consequently higher accuracy. For women, there was a significant increasing trend in model accuracy when the majority of recordings were taken on the second day (p=0.030), suggesting that consistent participation can improve model performance.

### Discussion

In this Example, the minimum number of voice recordings and improved recording settings required to maintain T2DM diagnostic efficacy was investigated in order to reduce patient burden. Our results showed that the model accuracy increased by including up to 6 voice recordings, with no significant improvement by including more voice samples. In both men and women, the variations in model accuracy across different days were not significantly different. However, adherence to recording in a single day showed higher accuracy.

Previous studies have shown that an increased burden from the treatment and self-management of chronic health conditions such as T2DM is associated with higher levels of distress, lower adherence to self-care routines, decreased satisfaction with medications, reduced quality of life, poorer physical and mental health, and greater risk of complications and deaths (J.S. Gonzalez, Eton, Janoo, Chen). In alignment with these, our study demonstrated that up to 6 voice recordings are sufficient to effectively diagnose T2DM, thereby reducing patient burden while maintaining diagnostic accuracy.

There are conflicting reports on self-management among men and women. While (Zhou) observed that women exhibited more compliance in self-care than men, (Mogre) reported higher self-monitoring of blood glucose in men than women. In our study, while the number of women were lower than men participants, women showed higher adherence to voice recording protocol than men. This higher adherence among women could explain the observed increase in model accuracy for women as the recordings were distributed across two days, indicating that consistent participation may enhance model performance.

Prior research has demonstrated that there is no significant day-to-day variability in voice recordings while there exists a significant time-of-day influence on acoustics with voice quality enhanced with increased voice use (Heald, Pierce). In alignment with these findings, our results showed that both in men and women, the accuracy of the model was not significantly different between days while there is an increase in accuracy when the majority of the recordings are from a single day.

One of the limitations of the current study is the low sample size for the women participants, which may restrict the generalizability of the findings and limit statistical power for detecting sex-specific effects or differences. Future studies are required to confirm these observations with larger and more diverse samples of female participants.

### CONCLUSION

This study is the first, to the best of our knowledge, to explore the improved balance between the number of voice recordings and recording settings, aiming for reduced patient's burden while maintaining diagnostic efficacy. By focusing on reducing the demands on patients, this Example aims to make T2DM screening more accessible through enhanced patient compliance and comfort. By demonstrating the feasibility of minimizing recording requirements without compromising diagnostic efficacy, this study opens avenues for future research and clinical applications aimed at optimizing healthcare delivery for T2DM.

### REFERENCES

Bommer C, Sagalova V, Heesemann E, Manne-Goehler J, Atun R, Bärnighausen T, Davies J, and Vollmer S. Global Economic Burden of Diabetes in Adults: Projections From 2015 to 2030. Diabetes Care 2018 February; 41:963-70. DOI:10.2337/dc17-1962. eprint: https://diabetesjournals.org/care/article-pdf/41/5/963/553413/dc171962.pdf. Available from: https://doi.org/10.2337/dc17-1962.
Harding J L, Pavkov M E, Magliano D J, Shaw J E, and Gregg E W. Global trends in diabetes complications: a review of current evidence. Diabetologia 2019; 62:3-16.
Zhang Z. Mechanics of human voice production and control. The journal of the acoustical society of America 2016; 140:2614-35.
Alam Z, Simonetti A, Brillantino R, Tayler N, Grainge C, Siribaddana P, Nouraei S, Batchelor J, Rahman M S, Mancuzo E V, et al. Predicting pulmonary function from the analysis of voice: a machine learning approach. Frontiers in digital health 2022; 4:5.
Rosen C A, Anderson D, and Murry T. Evaluating hoarseness: keeping your patient's voice healthy. American family physician 1998; 57:2775.
Sara J D S, Maor E, Orbelo D, Gulati R, Lerman L O, and Lerman A. Noninvasive voice biomarker is associated with incident coronary artery disease events at follow-up. Mayo Clinic Proceedings. Vol. 97. 5. Elsevier. 2022:835-46.
Roy N, Merrill R M, Pierce J, and Sundar K M. Voice disorders in obstructive sleep apnea: Prevalence, risk factors, and the role of CPAP. Annals of Otology, Rhinology & Laryngology 2019; 128:249-62.
López-de-Ipiña K, Martinez-de-Lizarduy U, Calvo P M, Beitia B, Garcia-Melero J, Fernández E, Ecay-Torres M, Faundez-Zanuy M, and Sanz P. On the analysis of speech and disfluencies for automatic detection of Mild Cognitive Impairment. Neural Computing and Applications 2020; 32:15761-9.
Wang J, Zhang L, Liu T, Pan W, Hu B, and Zhu T. Acoustic differences between healthy and depressed people: a cross-situation study. BMC psychiatry 2019; 19:1-12.
Yagihashi S, Mizukami H, and Sugimoto K. Mechanism of diabetic neuropathy: where are we now and where to go? Journal of diabetes investigation 2011; 2:18-32.
Ciarambino T, Crispino P, Leto G, Mastrolorenzo E, Para O, and Giordano M. Influence of gender in diabetes mellitus and its complication. International Journal of Molecular Sciences 2022; 23:8850.
Palomo-Osuna J, Failde I, De Sola H, and Dueñas M. Differences in Cognitive Function in Women and Men with Diabetic Peripheral Neuropathy with or without Pain. International Journal of Environmental Research and Public Health 2022; 19:17102.
Pinyopodjanard S, Suppakitjanusant P, Lomprew P, Kasemkosin N, Chailurkit L, and Ongphiphadhanakul B. Instrumental acoustic voice characteristics in adults with type 2 diabetes. Journal of Voice 2021; 35:116-21.
Hamdan Al, Jabbour J, Nassar J, Dahouk I, and Azar S T. Vocal characteristics in patients with type 2 diabetes mellitus. European Archives of Oto-Rhino-Laryngology 2012; 269:1489-95.
Gölaç H, Atalik G, Türkcan A K, and Yilmaz M. Disease related changes in vocal parameters of patients with type 2 diabetes mellitus. Logopedics Phoniatrics Vocology 2022; 47:202-8.
Chitkara D and Sharma R. Voice based detection of type 2 diabetes mellitus. 2016 2nd International Conference on Advances in Electrical, Electronics, Information, Communication and Bio-Informatics (AEEICB). IEEE. 2016:83-7.
Sidorova, P. Carbonell, and M. Cukic, “Blood Glucose Estimation From Voice: First Review of Successes and Challenges,” J Voice, vol. 36, no. 5, pp. 737 e1-737 e10, September 2022, doi: 10.1016/j.jvoice.2020.08.034.American Diabetes Association Professional Practice Committee. Classification and diagnosis of diabetes: standards of medical care in diabetes-2022. Diabetes Care 2022; 45: S17-S38. DOI:10.2337/dc22-S002. Available from: https://doi.org/10.2337/dc22-S002.
Type 2 diabetes glucose biomarker study with a continuous glucose monitoring system. Clinicaltrials.gov identifier: NCT04529239. Updated. Available from: https://clinicaltrials.gov/ct2/show/NCT04529239.
Jadoul Y, Thompson B, and Boer B de. Introducing Parselmouth: A Python interface to Praat. Journal of Phonetics 2018; 71:1-15. DOI:10.1016/j.wocn.2018.07.001. Available from: https://doi.org/10.1016/j.wocn.2018.07.001.
Boersma P and Weenink D. Praat, a system for doing phonetics by computer, version 3.4. Report 132. Institute of Phonetic Sciences of the University of Amsterdam, 1996:182.
Ancillao A, Galli M, Mignano M, Dellavalle R, Albertini G, et al. Quantitative analysis of pathological female human voice by processing complete sentences recordings. Journal of Laryngology and Voice 2013; 3:46.
Daousi C, Casson I F, Gill G V, MacFarlane I A, Wilding J P, and Pinkney J H. Prevalence of obesity in type 2 diabetes in secondary care: association with cardiovascular risk factors. Postgrad Med J 2006 April; 82:280-4. DOI:10.1136/pmj.2005.039032
Tandon N, Anjana R M, Mohan V, Kaur T, Afshin A, Ong K, Mukhopadhyay S, Thomas N, Bhatia E, Krishnan A, et al. The increasing burden of diabetes and variations among the states of India: the Global Burden of Disease Study 1990-2016. The Lancet Global Health 2018; 6: e1352-e1362.
Ravishankar S, Kumar M. K. P, Patage V V, Tiwari S, and Goyal S. Prediction of Age from Speech Features Using a Multi-Layer Perceptron Model. 2020 11th International Conference on Computing, Communication and Networking Technologies (ICCCNT). 2020:1-6. DOI:10.1109/ICCCNT49239.2020.9225390.
R. Goyal, M. Singhal, and I. Jialal, “Type 2 Diabetes,” in StatPearls. Treasure Island (F L), 2024.
Souza L B R and Santos M M D. Body mass index and acoustic voice parameters: is there a relationship? Braz J Otorhinolaryngol 2018 July; 84 Epub 2017 May 6:410-5. DOI: 10.1016/j.bjorl.2017.04.003.
Perry B D, Caldow M K, Brennan-Speranza T C, Sbaraglia M, Jerums G, Garnham A, Wong C, Levinger P, Haq M A ul, Hare D L, et al. Muscle atrophy in patients with Type 2 Diabetes Mellitus: roles of inflammatory pathways, physical activity and exercise. Exercise immunology review 2016; 22:94.
Hicks C W and Selvin E. Epidemiology of peripheral neuropathy and lower extremity disease in diabetes. Current diabetes reports 2019; 19:1-8.
Deischinger C, Dervic E, Leutner M, Kosi-Trebotic L, Klimek P, Kautzky A, and Kautzky-Willer A. Diabetes mellitus is associated with a higher risk for major depressive disorder in women than in men. BMJ Open Diabetes Research and Care 2020; 8: e001430.
Eyben F, Wöllmer M, and Schuller B. Opensmile: The Munich Versatile and Fast Open-Source Audio Feature Extractor. Proceedings of the 18th ACM International Conference on Multimedia. M M '10. Firenze, Italy: Association for Computing Machinery, 2010:1459-62. DOI:10.1145/1873951.1874246. Available from: https://doi.org/10.1145/1873951.1874246
Alvi G B, Qadir M I, Ali B. Assessment of Inter-Connection between Suri phobia and Individual's Blood Glucose Level: A Questionnaire Centred Project. J Clin Exp Immunol 2019; 4.
Bailey T, Bode B W, Christiansen M P, Klaff L J, Alva S. The Performance and Usability of a Factory-Calibrated Flash Glucose Monitoring System. Diabetes Technol Ther 2015. DOI:10.1089/dia.2014.0378.
Beagley J, Guariguata L, Weil C, Motala A A. Global estimates of undiagnosed diabetes in adults. Diabetes Res Clin Pract 2014. DOI:10.1016/j.diabres.2013.11.001.
Bonneh Y S, Levanon Y, Dean-Pardo O, Lossos L, Adini Y. Abnormal speech spectrum and increased pitch variability in young autistic children. Front Hum Neurosci 2011. DOI:10.3389/fnhum.2010.00237.
Chen, F. A. Sloan, and A. P. Yashkin, “Adherence to diabetes guidelines for screening, physical activity and medication and onset of complications and death,” Journal of Diabetes and its Complications, vol. 29, no. 8, pp. 1228-1233, 2015.
Colton R H, Casper J K, Leonard R. Understanding voice problem: A physiological perspective for diagnosis and treatment: Fourth edition. 2011.
Czupryniak L, Sielska-Badurek E, Agnieszka N, et al. 378-P: Human Voice Is Modulated by Hypoglycemia and Hyperglycemia in Type 1 Diabetes. Am Diabetes Assoc San Fr Calif (poster Present 2019.
Daniel P M, Love E R, Pratt O E. Insulin-stimulated entry of glucose into muscle in vivo as a major factor in the regulation of blood glucose. J Physiol 1975. DOI:10.1113/jphysiol. 1975.sp010931
Eskidere O, Gürhanli A. Voice Disorder Classification Based on Multitaper MeI Frequency Cepstral Coefficients Features. Comput Math Methods Med 2015. DOI:10.1155/2015/956249.
Eton et al., “Development and validation of the Patient Experience with Treatment and Self-management (PETS): a patient-reported measure of treatment burden,” Quality of Life Research, vol. 26, pp. 489-503, 2017.
Eyben F, Wollmer M, Schuller B B, Weninger F, Wollmer M, Schuller B B. OPENSMILE: open-Source Media Interpretation by Large feature-space Extraction. MM′10-Proc ACM Multimed 2010 Int Conf 2015. DOI:10.1145/1873951.1874246.
Francisco-García V, Guzmán-Guzmán I P, Salgado-Rivera R, Alonso-Silverio G A, Alarcón-Paredes A. Non-invasive Glucose Level Estimation: A Comparison of Regression Models Using the MFCC as Feature Extractor. In: Lecture Notes in Computer Science (including subseries Lecture Notes in Artificial Intelligence and Lecture Notes in Bioinformatics). 2019. DOI:10.1007/978-3-030-21077-9_19.
Fraser K C, Meltzer J A, Rudzicz F. Linguistic features identify Alzheimer's disease in narrative speech. J Alzheimer's Dis 2015. DOI:10.3233/JAD-150520.
Hamdan A L, Jabbour J, Nassar J, Dahouk I, Azar S T. Vocal characteristics in patients with type 2 diabetes mellitus. Eur Arch Oto-Rhino-Laryngology 2012. DOI:10.1007/s00405-012-1933-7.
Hamdan A L, Dowli A, Barazi R, Jabbour J, Azar S. Laryngeal sensory neuropathy in patients with diabetes mellitus. J Laryngol Otol 2014. DOI:10.1017/S002221511400139X.
Hamdan, Z. Kurban, and S. T. Azar, “Prevalence of phonatory symptoms in patients with type 2 diabetes mellitus,” Acta Diabetol, vol. 50, no. 5, pp. 731-6, October 2013, doi: 10.1007/s00592-012-0392-3.
Hari Kumar K V S, Garg A, Ajai Chandra N S, Singh S P, Datta R. Voice and endocrinology. Indian J. Endocrinol. Metab. 2016. DOI:10.4103/2230-8210.190523.
Heald and H. C. Nusbaum, “Variability in vowel production within and between days,” PLOS One, vol. 10, no. 9, p. e0136791, 2015.
Holl R W, Heinze E. Dawn or Somogyi phenomenon? High morning fasting blood sugar levels in juvenile type 1 diabetics. Dtsch Medizinische Wochenschrift 1992. DOI:10.1055/s-2008-1062470.
Hoseini A, Mirzapour A, Bijani A, Shirzad A. Salivary flow rate and xerostomia in patients with type I and II diabetes mellitus. Electron Physician 2017. DOI:10.19082/5244.
Hoss U, Budiman E S, Liu H, Christiansen M P. Continuous glucose monitoring in the subcutaneous tissue over a 14-day sensor wear period. J Diabetes Sci Technol 2013. DOI:10.1177/193229681300700511.
Hsu H Y, Chiu H Y, Lin H T, Su F C, Lu C H, Kuo L C. Impacts of elevated glycemic haemoglobin and disease duration on the sensorimotor control of hands in diabetes patients. Diabetes Metab Res Rev 2015. DOI:10.1002/dmrr.2623.
Jackson R, Brennan S, Fielding P, et al. Distinct and complementary roles for a and B isoenzymes of PKC in mediating vasoconstrictor responses to acutely elevated glucose. Br J Pharmacol 2016. DOI:10.1111/bph. 13399.
J. S. Gonzalez, M. L. Tanenbaum, and P. V. Commissariat, “Psychosocial factors in medication adherence and diabetes self-management: Implications for research and practice,” American Psychologist, vol. 71, no. 7, p. 539, 2016.
Jannoo and N. M. Khan, “Medication adherence and diabetes self-care activities among patients with type 2 diabetes mellitus,” Value in health regional issues, vol. 18, pp. 30-35, 2019.
Kaufman, A. Thommandram, and Y. Fossat, “Acoustic analysis and prediction of type 2 diabetes mellitus using smartphone-recorded voice segments,” Mayo Clinic Proceedings: Digital Health, vol. 1, no. 4, pp. 534-544, 2023.
Kirchberger M, Russo F A. Dynamic Range Across Music Genres and the Perception of Dynamic Compression in Hearing-Impaired Listeners. In: Trends in Hearing. 2016. DOI:10.1177/2331216516630549.
Koo T K, Li M Y. A Guideline of Selecting and Reporting Intraclass Correlation Coefficients for Reliability Research. J Chiropr Med 2016. DOI:10.1016/j.jcm.2016.02.012.
Malouf R, Brust J C M. Hypoglycemia: Causes, neurological manifestations, and outcome. Ann. Neurol. 1985. DOI:10.1002/ana.410170502.
Maor E, Perry D, Mevorach D, et al. Vocal Biomarker Is Associated With Hospitalization and Mortality Among Heart Failure Patients. J Am Heart Assoc 2020. DOI:10.1161/JAHA. 119.013359.
Marmar C R, Brown A D, Qian M, et al. Speech-based markers for posttraumatic stress disorder in U S veterans. Depress Anxiety 2019. DOI:10.1002/da.22890.
Mogre, Z. O. Abanga, F. Tzelepis, N. A. Johnson, and C. Paul, “Adherence to and factors associated with self-care behaviours in type 2 diabetes patients in Ghana,” BMC Endocrine Disorders, vol. 17, pp. 1-8, 2017.
Noffs G, Perera T, Kolbe S C, et al. What speech can tell us: A systematic review of dysarthria characteristics in Multiple Sclerosis. Autoimmun. Rev. 2018. DOI:10.1016/j.autrev.2018.06.010.
Pinyopodjanard S, Suppakitjanusant P, Lomprew P, Kasemkosin N, Chailurkit L, Ongphiphadhanakul B. Instrumental Acoustic Voice Characteristics in Adults with Type 2 Diabetes. J Voice 2019. DOI:10.1016/j.jvoice.2019.07.003.
P'ng C, Green J, Chong L C, et al. BPG: Seamless, automated and interactive visualization of scientific data. BMC Bioinformatics 2019. DOI:10.1186/s12859-019-2610-2.
Pierce, K. Tanner, R. M. Merrill, L. Shnowske, and N. Roy, “Acoustic variability in the healthy female voice within and across days: how much and why?,” Journal of Speech, Language, and Hearing Research, vol. 64, no. 8, pp. 3015-3031, 2021.
Ribeiro M T, Singh S, Guestrin C. ‘Why should i trust you?’ Explaining the predictions of any classifier. In: Proceedings of the ACM SIGKDD International Conference on Knowledge Discovery and Data Mining. 2016. DOI:10.1145/2939672.2939778.
Sivasankar M, Leydon C. The role of hydration in vocal fold physiology. Curr. Opin. Otolaryngol. Head Neck Surg. 2010. DOI:10.1097/MOO.0b013e3283393784.
Standards of medical care for patients with diabetes mellitus. Diabetes Care. 2003. DOI:10.2337/diacare.26.2007.s33.
Statistics About Diabetes. https://www.diabetes.org/resources/statistics/statistics-about-diabetes.
H. Sun et al., “IDF Diabetes Atlas: Global, regional and country-level diabetes prevalence estimates for 2021 and projections for 2045,” Diabetes research and clinical practice, vol. 183, p. 109119, 2022.
Vaiciukynas E, Verikas A, Gelzinis A, Bacauskiene M. Detecting Parkinson's disease from sustained phonation and speech signals. PLoS One 2017. DOI:10.1371/journal.pone.0185613.
Veen L van, Morra J, Palanica A, Fossat Y. Homeostasis as a proportional-integral control system. npj Digit Med 2020. DOI:10.1038/s41746-020-0283-x.
V. Mogre, N. A. Johnson, F. Tzelepis, and C. Paul, “Barriers to diabetic self-care: A qualitative study of patients' and healthcare providers' perspectives,” Journal of clinical nursing, vol. 28, no. 11-12, pp. 2296-2308, 2019.
Weinreb, K. Piersiala, A. T. Hillel, L. M. Akst, and S. R. Best, “Dysphonia and dysphagia as early manifestations of autoimmune inflammatory myopathy,” American journal of otolaryngology, vol. 42, no. 1, p. 102747, 2021.
Wild S H, Smith F B, Lee A J, Fowkes F G R. Criteria for previously undiagnosed diabetes and risk of mortality: 15-Year follow-up of the Edinburgh Artery Study cohort. Diabet Med 2005. DOI:10.1111/j.1464-5491.2004.01433.x.
Zhang Y, Santosa A, Wang N, et al. Prevalence and the Association of Body Mass Index and Other Risk Factors with Prediabetes and Type 2 Diabetes Among 50,867 Adults in China and Sweden: A Cross-Sectional Study. Diabetes Ther 2019. DOI:10.1007/s13300-019-00690-3.
Zhou et al., “Relationship between self-care compliance, trust, and satisfaction among hypertensive patients in China,” Frontiers in Public Health, vol. 10, p. 1085047, 2023.

1. A computer-implemented method for generating a type-II (T2DM) diabetic status prediction model, the method comprising:
providing, at a memory:
a plurality of diabetic status labels identifying a corresponding diabetic status for a plurality of training subjects; and
a plurality of voice samples collected from the plurality of training subjects at a plurality of time points, each of the voice samples in the plurality of voice samples associated with a corresponding diabetic status label in the plurality of diabetic status labels;
determining, at the processor, a plurality of voice feature values for a corresponding plurality of voice features for each of the voice samples in the plurality of voice samples; and
generating, at the processor, the type-II (T2DM) diabetic status prediction model based on the plurality of voice samples and the plurality of voice feature values.

2. The method of claim 1, wherein each of the at least one voice biomarker feature value may be selected from the group comprising: a statistical feature category, a shimmer feature category, and a jitter feature category.

3. The method of claim 2, wherein:
the statistical feature category may comprise a mean pitch feature value, a pitch standard deviation feature value, a mean intensity feature value, an intensity standard deviation feature value and a harmonic-to-noise ratio feature value;
the shimmer feature category may comprise a localShimmer feature value, a localdbShimmer feature value, an apq3Shimmer feature value, an apq5Shimmer feature value, and an apq11Shimmer feature value; and
the jitter feature category may comprise a localJitter feature value, a localabsJitter feature value, a rapJitter feature value and a ppq5Jitter feature value.

4. The method of claim 1, further comprising:
selecting, at the processor, a subset of voice feature values from the plurality of voice feature values for each voice sample based on a Cohen's d effect size of each voice feature value; and
wherein the generating at the processor, the type-II (T2DM) diabetic status prediction model is based on the plurality of voice samples and the subset of voice feature values.

5. The method of claim 4 further comprising:
preprocessing, at the processor, each voice sample by averaging the voice sample based on at least one of the plurality of historical voice samples of the subject.

6. The method of claim 5, wherein the plurality of voice samples may comprise an age and BMI matched dataset comprising voice samples from an equal number of T2DM subjects and non-diabetic subjects.

7. The method of claim 6, wherein each voice sample may comprise a predetermined phrase vocalized by the corresponding subject.

8. The method of claim 7, wherein the predetermined phrase may be displayed to the subject on a display device of a user device and each voice sample may be recorded using an audio input device of the user device.

9. The method of claim 8, wherein the memory may further comprise:
at least one selected from the group of: vocal parameter data for the plurality of subjects, age data of the plurality of subjects, and Body Mass Index (BMI) data of the plurality of subjects; and
wherein the generating at the processor, the type-II (T2DM) diabetic status prediction model may be further based on the at least one selected from the group of: vocal parameter data for the plurality of subjects, age data of the plurality of subjects, and Body Mass Index (BMI) data of the plurality of subjects.

10. The method of claim 9, wherein the type-II (T2DM) diabetic status prediction model may comprise at least one selected from the group of a Logistic Regression (LR) model, a Naïve Bayes (NB) model, and a Support Vector Machine (SVM) model.

11. A computer-implemented system for generating a type-II (T2DM) diabetic status prediction model, the method comprising:
a memory comprising:
a plurality of diabetic status labels identifying a corresponding diabetic status for a plurality of training subjects; and
a plurality of voice samples collected from the plurality of training subjects at a plurality of time points, each of the voice samples in the plurality of voice samples associated with a corresponding diabetic status label in the plurality of diabetic status labels;
a processor in communication with the memory, the processor configured to:
determine a plurality of voice feature values for a corresponding plurality of voice features for each of the voice samples in the plurality of voice samples; and
generate the type-II (T2DM) diabetic status prediction model based on the plurality of voice samples and the plurality of voice feature values.

12. The system of claim 11, wherein each of the at least one voice biomarker feature value may be selected from the group comprising: a statistical feature category, a shimmer feature category, and a jitter feature category.

13. The system of claim 12, wherein:
the statistical feature category may comprise a mean pitch feature value, a pitch standard deviation feature value, a mean intensity feature value, an intensity standard deviation feature value and a harmonic-to-noise ratio feature value;
the shimmer feature category may comprise a localShimmer feature value, a localdbShimmer feature value, an apq3Shimmer feature value, an apq5Shimmer feature value, and an apq11Shimmer feature value; and
the jitter feature category may comprise a localJitter feature value, a localabsJitter feature value, a rapJitter feature value and a ppq5Jitter feature value.

14. The system of claim 11, where the processor if further configured to:
select a subset of voice feature values from the plurality of voice feature values for each voice sample based on a Cohen's d effect size of each voice feature value; and
wherein the type-II (T2DM) diabetic status prediction model is based on the plurality of voice samples and the subset of voice feature values.

15. The system of claim 14, wherein the processor is further configured to:
preprocess each voice sample by averaging the voice sample based on at least one of the plurality of historical voice samples of the subject.

16. The system of claim 15, wherein the plurality of voice samples may comprise an age and BMI matched dataset comprising voice samples from an equal number of T2DM subjects and non-diabetic subjects.

17. The system of claim 16, wherein each voice sample may comprise a predetermined phrase vocalized by the corresponding subject.

18. The system of claim 17, wherein the predetermined phrase may be displayed to the subject on a display device of a user device and each voice sample may be recorded using an audio input device of the user device.

19. The system of claim 18, wherein the memory may further comprise:
at least one selected from the group of: vocal parameter data for the plurality of subjects, age data of the plurality of subjects, and Body Mass Index (BMI) data of the plurality of subjects; and
wherein the type-II (T2DM) diabetic status prediction model may be further based on the at least one selected from the group of: vocal parameter data for the plurality of subjects, age data of the plurality of subjects, and Body Mass Index (BMI) data of the plurality of subjects.

20. The system of claim 19, wherein the type-II (T2DM) diabetic status prediction model may comprise at least one selected from the group of a Logistic Regression (LR) model, a Naïve Bayes (NB) model, and a Support Vector Machine (SVM) model.
